(19) United States
(12) Patent Application Publication
(10) Pub. No.: US 2026/0083854 A1
(43) Pub. Date: Mar. 26, 2026

(54) CONJUGATES COMPRISING BIS-MSPT LINKER

(71) Applicant: ELI LILLY AND COMPANY, Indianapolis, IN (US)

(72) Inventors: Riazul ALAM, Fishers, IN (US); Guillermo S. CORTEZ, Indianapolis, IN (US); Yan WANG, Carmel, IN (US)

(21) Appl. No.: 19/333,454

(22) Filed: Sep. 19, 2025

Related U.S. Application Data
(60) Provisional application No. 63/698,736, filed on Sep. 25, 2024.

(51) Int. Cl. A61K 47/68; A61K 9/00; A61P 25/28; C07K 16/28

(52) U.S. Cl. CPC A61K 47/6807; A61K 9/0019; A61K 47/6849; A61K 47/6889; A61P 25/28; C07K 16/2881

(57) ABSTRACT

Provided herein are antibody conjugates comprising a bis-MSPT linker, pharmaceutical compositions comprising such conjugates, and methods of making or using such conjugates.

### SEQUENCE LISTING

The present application is being filed along with a Sequence Listing in ST.26 XML format. The Sequence Listing is provided as a file titled “31061_US” created Aug. 26, 2025, and is 98,486 bytes in size. The Sequence Listing information in the ST.26 XML format is incorporated herein by reference in its entirety.

### BACKGROUND

Antibody drug conjugates can be used to deliver therapeutic agents such as oligonucleotides to specific tissues and achieve gene silencing or activation in the target tissue. For example, WO 2024/036096 describes transferrin receptor (“TfR”) binding proteins and conjugates for delivering therapeutic agents such as oligonucleotides across blood brain barrier. However, the process of making such antibody conjugates can involve multiple steps and be time consuming. Linkers can play an important role in making antibody conjugates and impact the conjugate stability and activity.

Therefore, there remains a need for improving antibody conjugates, linker chemistry, and the process of making such conjugates.

### SUMMARY OF INVENTION

Provided herein are conjugates comprising a bis-MSPT (4-(5-methylsulfonyl-1H-tetrazole-1yl) phenol) linker, pharmaceutical compositions comprising such conjugates, and methods of making or using such conjugates. The conjugates provided herein have simplified manufacturing process and/or improved plasma stability.

In one aspect, provided herein are conjugates of Formula (I): A-L-D, wherein A is an antibody or antibody fragment comprising two cysteine residues, wherein D is a therapeutic agent, and wherein L is a linker comprising the following formula:

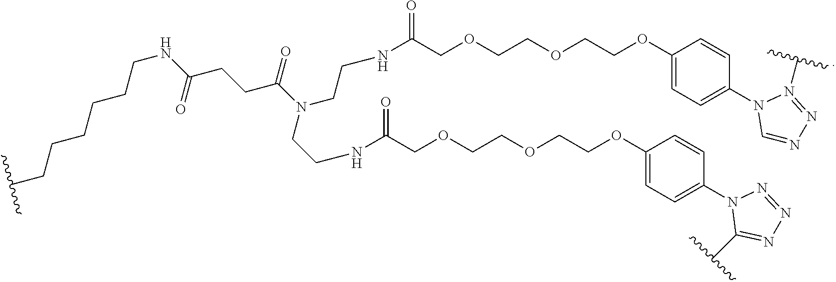

In some embodiments, provided herein are conjugates comprising the following formula:

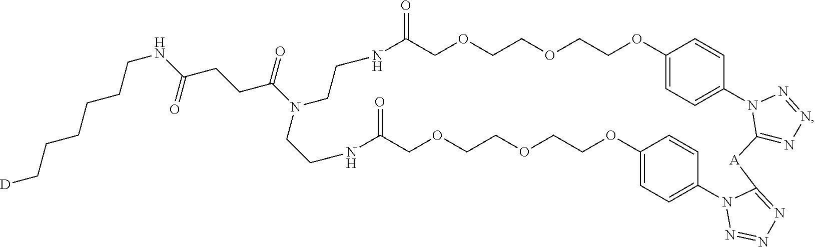

wherein A is an antibody or antibody fragment comprising two cysteine residues, and wherein D is a therapeutic agent.

The conjugates provided herein have simplified manufacturing process and/or improved plasma stability.

In some embodiments, A is a monoclonal antibody, heterodimeric antibody, one-arm heteromab, Fab, or Fab-VHH.

In some embodiments, D is an oligonucleotide (e.g., antisense oligonucleotide), double stranded RNA (e.g., siRNA, saRNA), polypeptide, small molecule, nanoparticle, lipid nanoparticle, exosome, antibody or antigen binding fragment thereof, or a combination thereof. In some embodiments, the therapeutic agent is an oligonucleotide. In some embodiments, the therapeutic agent is a double stranded RNA (dsRNA), e.g., a dsRNA comprising a sense stand and an antisense strand.

In some embodiments, the dsRNA comprises an antisense strand complementary to a target mRNA selected from SNCA, MAPT, APP, ATXN2, ATXN3, SARM1, APOE, BACE1, FMR1, LRRK2, HTT, SOD1, SCN10A, SCN9A, CACNA1B, or PRNP mRNA. In some embodiments, the dsRNA comprises an antisense strand complementary to SNCA mRNA.

Exemplary unmodified sense strand and antisense strand sequences of dsRNA targeting human SNCA mRNA are provided in Table 3a. In some embodiments, the sense strand and the antisense strand of the dsRNA comprise a pair of nucleic acid sequences selected from the group consisting of:
(a) the sense strand comprises SEQ ID NO: 23, and the antisense strand comprises SEQ ID NO: 24;
(b) the sense strand comprises SEQ ID NO: 25, and the antisense strand comprises SEQ ID NO: 24; and
(c) the sense strand comprises SEQ ID NO: 26, and the antisense strand comprises SEQ ID NO: 27,
   wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 23, and the antisense strand comprises SEQ ID NO: 24.

The dsRNA can include modifications. The modifications can be made to one or more nucleotides of the sense and/or antisense strand or to the internucleotide linkages. In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl (e.g., 2′-O—C₁₆ alkyl) modified nucleotide.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SNCA mRNA are provided in Table 3b.

In another aspect, provided herein are methods of treating a neurodegenerative disease, in a patient in need thereof, and such the method comprises administering to the patient an effective amount of a conjugate or a pharmaceutical composition described herein.

In some embodiments, the neurodegenerative disease is Parkinson's disease, Alzheimer's disease, multiple system atrophy, or Lewy body dementia.

The conjugate or a pharmaceutical composition can be administered to the patient intravenously or subcutaneously.

In another aspect, provided herein are conjugates described herein or pharmaceutical compositions comprising such conjugates for use in a therapy. Also provided herein are conjugates described herein or pharmaceutical compositions comprising such conjugates for use in the treatment of a neurodegenerative disease.

In another aspect, provided herein are compounds comprising the following formula:

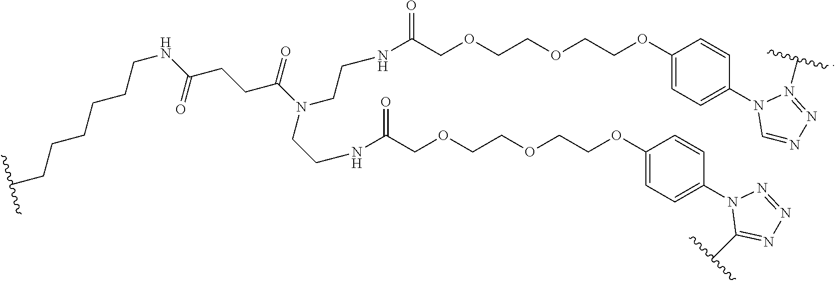

In some embodiments, provided herein are compounds comprising the following formula:

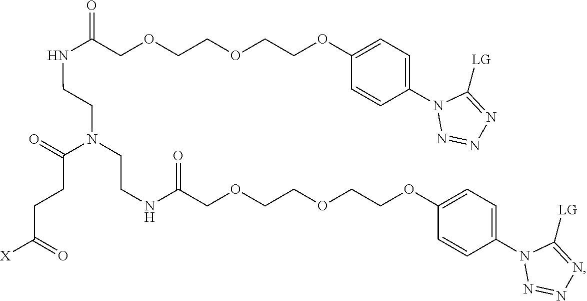

wherein LG is a leaving group and X is an amide coupling partner.

In some embodiments, provided herein are compounds comprising the following formula:

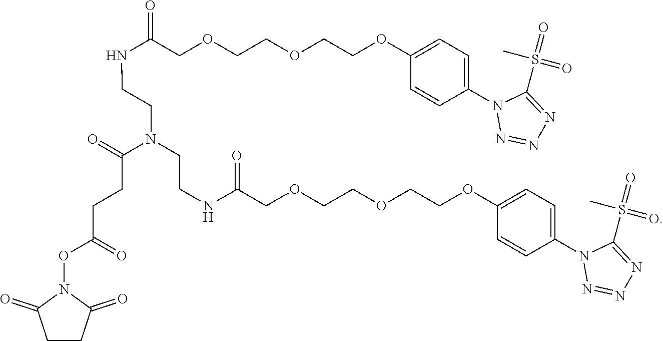 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

In another aspect, provided herein are methods of generating a conjugate, such methods comprising reacting an antibody comprising two cysteine residues with a compound comprising the following formula:

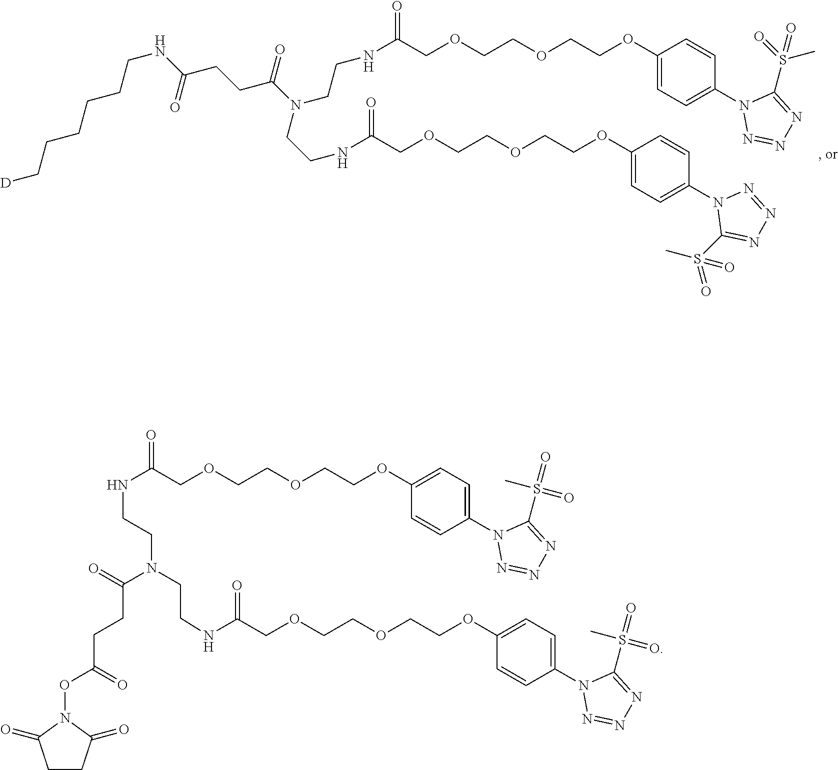 [SMILES: [2H]CCCCCCNC(=O)CCC(=O)N(CCNC(=O)COCCOCCOc1ccc(-n2nnnc2S(C)(=O)=O)cc1)CCNC(=O)COCCOCCOc1ccc(-n2nnnc2S(C)(=O)=O)cc1 ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

### BRIEF DESCRIPTION OF THE DRAWINGS

FIG. 1A shows exemplary diagrams of conjugates comprising a bis-MSPT linker described herein. FIG. 1B shows exemplary diagrams of conjugates comprising mono-linkers. FIG. 1C shows the process of making conjugates comprising mono-MSPT linkers. FIG. 1D shows the improved/simplified process of making conjugates comprising bis-MSPT linkers described herein.

FIGS. 2A and 2B show exemplary analytical anion exchange (AEX) chromatogram of DAR profile for A4-MSPT-dsRNA No. 4 conjugate before (2A) or after (2B) purification. FIGS. 2C and 2D show exemplary analytical anion exchange (AEX) chromatogram of DAR profile for A5-bis-MSPT-dsRNA No. 4 conjugate before (2C) or after (2D) purification. FIGS. 2E and 2F show exemplary analytical anion exchange (AEX) chromatogram of DAR profile for A2-MSPT-dsRNA No. 4 conjugate before (2E) or after (2F) purification. FIGS. 2G and 2H show exemplary analytical anion exchange (AEX) chromatogram of DAR profile for A3-bis-MSPT-dsRNA No. 4 conjugate before (2G) or after (2H) purification.

FIG. 3 show SNCA mRNA reduction in human transferrin transgenic mouse brain 28 days following a single intravenous (IV) delivery of the indicated conjugates at 0.25 mg/kg siRNA dose. The error bars in FIG. 3 are Standard Deviations and statistical analysis was performed with a one-way ANOVA with Dunnett's multiple comparison test against PBS control group. Annotations indicate P values>0.0001=****; >0.001=***; >0.01=**; >0.05=*.

FIG. 4 shows siRNA payload stability across conjugates in mouse and cynomolgus monkey plasma over 48 hours.

### DETAILED DESCRIPTION

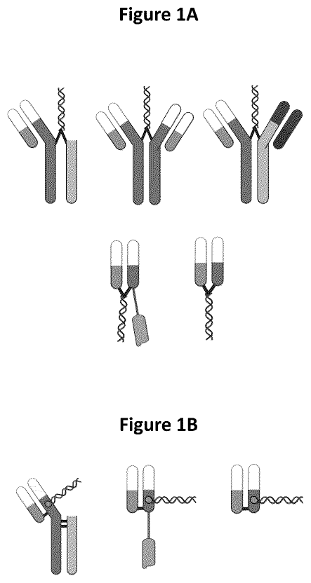

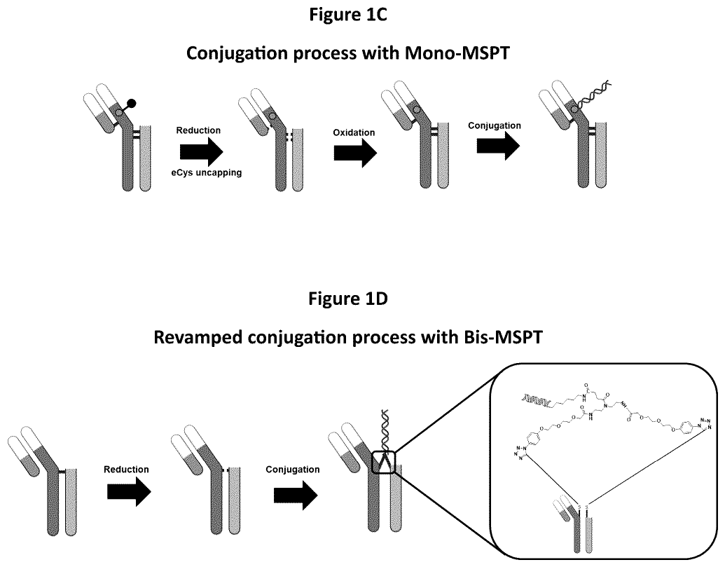

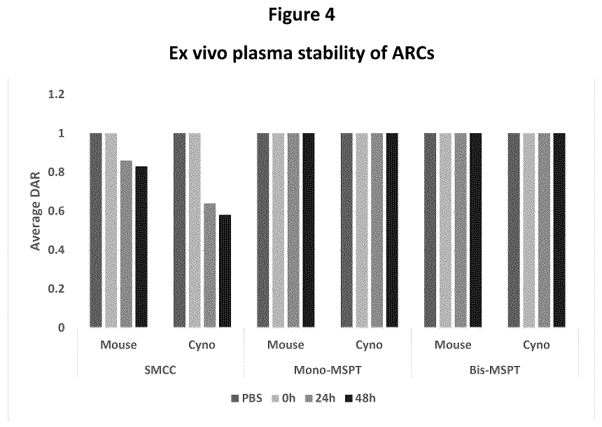

Provided herein are conjugates comprising a bis-MSPT linker, pharmaceutical compositions comprising such conjugates, and methods of making or using such conjugates. The conjugates provided herein have improved/simplified manufacturing process (see FIGS. 1C and 1D) and/or improved plasma stability (see FIG. 4). The improved process allows for streamlining of the conjugation, whereby separate reduction and re-oxidation steps of the antibody scaffold prior to conjugation of siRNA payload is eliminated. Conjugation via Bis-MSPT onto the disulfide pair of the antibody or antibody fragment is done through a simplified single step of reduction of the cysteine disulfide pair, followed by conjugation of the siRNA payload.

In one aspect, provided herein are conjugates of Formula (I): A-L-D, wherein A is an antibody or antibody fragment comprising two cysteine residues, wherein D is a therapeutic agent, and wherein L is a linker comprising the following formula:

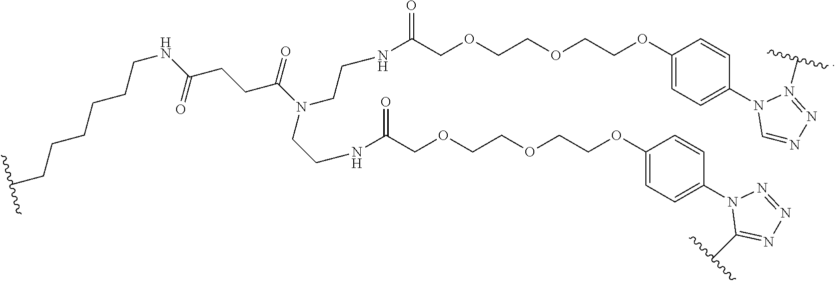

In some embodiments, provided herein are conjugates comprising the following formula:

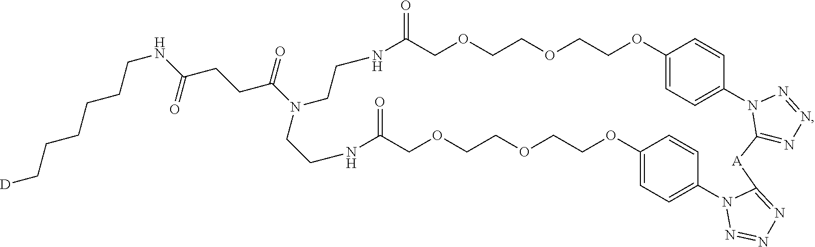

wherein A is an antibody or antibody fragment comprising two cysteine residues, and wherein D is a therapeutic agent.

In another aspect, provided herein are compounds comprising the following formula:

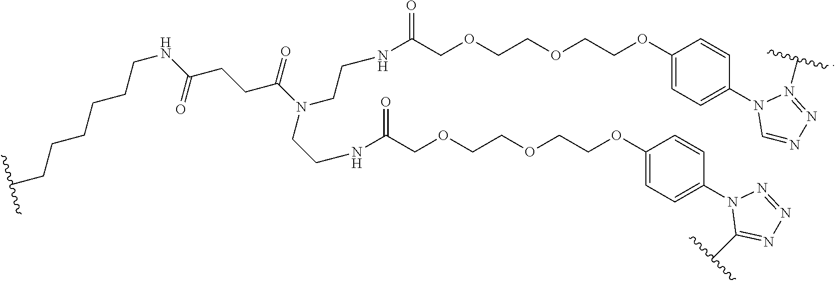

In some embodiments, provided herein are compounds comprising the following formula:

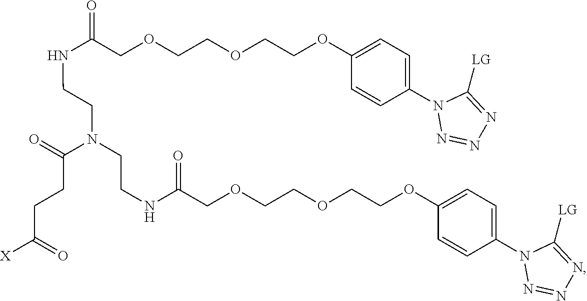

wherein LG is a leaving group and X is an amide coupling partner. A leaving group is a group of atoms that detaches from the main part of a substrate during a reaction. Commonly used leaving groups include halogens (i.e. I, Br, Cl, and F) and sulfonates (i.e. tosylate, mesylate, etc.). The amide coupling partner can include carboxylic acids, acid chlorides, or an activated ester such an NHS.

In some embodiments, provided herein are compounds comprising the following formula:

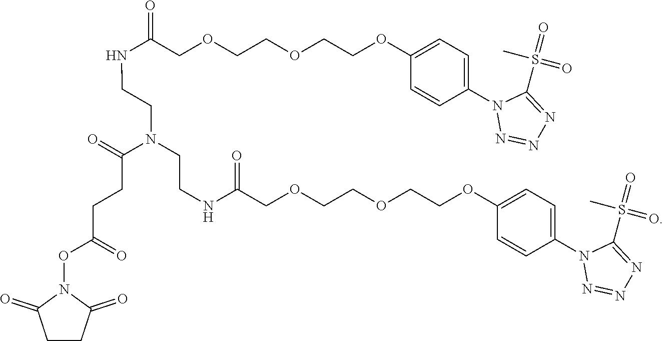 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

In another aspect, provided herein are methods of generating a conjugate, such methods comprising reacting an antibody comprising two cysteine residues with a compound comprising any one of the following formula:

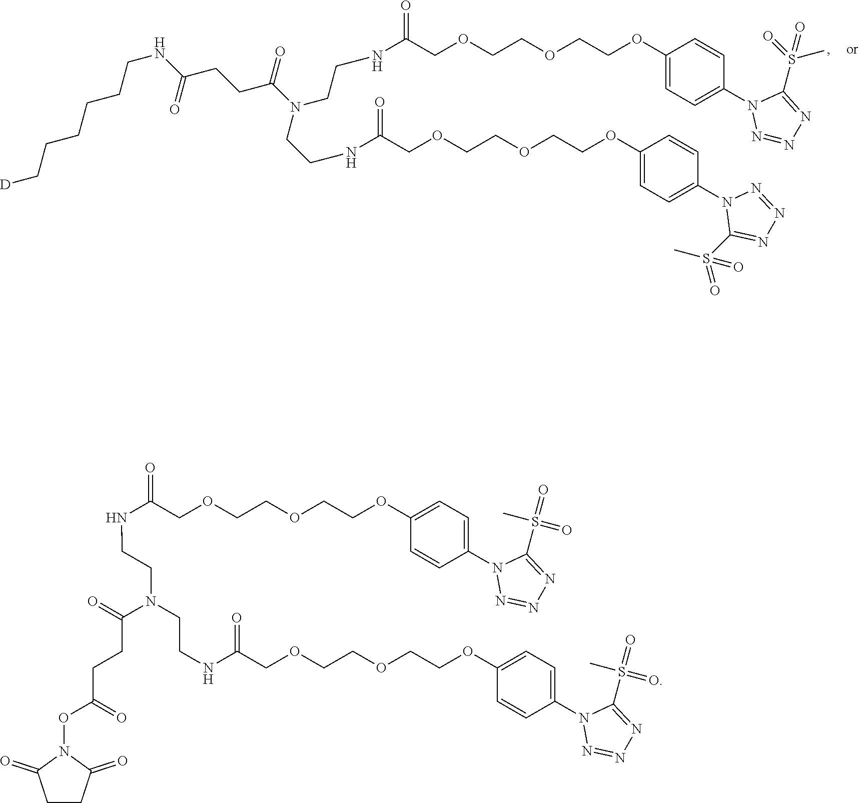 [SMILES: [2H]CCCCCCNC(=O)CCC(=O)N(CCNC(=O)COCCOCCOc1ccc(-n2nnnc2S(C)(=O)=O)cc1)CCNC(=O)COCCOCCOc1ccc(-n2nnnc2S(C)(=O)=O)cc1 ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

### Antibody or Antibody Fragment

The antibody or antibody fragment (A) of the present conjugates comprises two cysteine residues.

In some embodiments, A is a monoclonal antibody, heterodimeric antibody, one-arm heteromab (OAH), Fab, or Fab-VHH.

In some embodiments, A is an antibody or antibody fragment that binds human TfR (“human TfR binding antibody or antibody fragment”). Human TfR binding antibody or antibody fragment can bind TfR on BBB and transport therapeutic agent into the CNS.

Exemplary sequences of human TfR binding antibody or antibody fragment are provided in Table 1a and 1b. In some embodiments, the human TfR binding antibody or antibody fragment comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, VH comprises SEQ ID NO: 7, and VL comprises SEQ ID NO: 8. In some embodiments, VH comprises a sequence having at least 95% sequence identity to SEQ ID NO: 7, and VL comprises a sequence having at least 95% sequence identity to SEQ ID NO: 8.

TABLE 1a
Exemplary sequences of human
TfR binding antibody or antibody fragment

| | | SEQ |
|---|---|---|
| | | ID |
| Region | Sequence | NO |
| HCDR1 | SYSMN | 1 |
| (KABAT) | | |
| HCDR2 | SISSSSSYIYYADSVKG | 2 |
| (KABAT) | | |
| HCDR3 | RHGYSNSDAFDN | 3 |
| (KABAT) | | |
| LCDR1 | RASQGISHYLV | 4 |
| (KABAT) | | |
| LCDR2 | AASSLQS | 5 |
| (KABAT) | | |
| LCDR3 | LQHNSYPWT | 6 |
| (KABAT) | | |
| VH | EVQLVESGGGLVKPGGSLRLSCVASGFTFSSYS | 7 |
| | MNWVRQAPGKGLEWVSSISSSSSYIYYADSVKG | |
| | RFTISRDNAKNSLYLQMNSLRAEDTAVYYCARR | |
| | HGYSNSDAFDNWGQGTLVTVSS | |
| VL | DIQMTQSPSAMSASVGDRVTITCRASQGISHYL | 8 |
| | VWFQQKPGKVPKRLIYAASSLQSGVPSRFSGSG | |
| | SGTEFTLTISSLQPEDFATYYCLQHNSYPWTFG | |
| | QGTKVEIK | |
| Fab HC | EVQLVESGGGLVKPGGSLRLSCVASGFTFSSYS | 9 |
| | MNWVRQAPGKGLEWVSSISSSSSYIYYADSVKG | |
| | RFTISRDNAKNSLYLQMNSLRAEDTAVYYCARR | |
| | HGYSNSDAFDNWGQGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLG | |
| | TQTYICNVNHKPSNTKVDKRVEPKSC | |
| Fab-VHH1 | EVQLVESGGGLVKPGGSLRLSCVASGFTFSSYS | 10 |
| HC | MNWVRQAPGKGLEWVSSISSSSSYIYYADSVKG | |
| | RFTISRDNAKNSLYLQMNSLRAEDTAVYYCARR | |
| | HGYSNSDAFDNWGQGTLVTVSSASTKGPCVFPL | |
| | APSSKSTSGGTAALGCLVKDYFPEPVTVSWNSG | |
| | ALTSGVHTFPAVLQSSGLYSLSSVVTVPSSSLG | |
| | TQTYICNVNHKPSNTKVDKRVEPKSCDKTHTGG | |
| | GGQGGGGQGGGGQGGGGQGGGGQEVQLLESGGG | |
| | LVQPGGSLRLSCAASGRYIDETAVAWFRQAPGK | |
| | GREFVAGIGGGVDITYYADSVKGRFTISRDNSK | |
| | NTLYLQMNSLRPEDTAVYYCGARPGRPLITSKV | |
| | ADLYPYWGQGTLVTVSSPP | |
| Fab/Fab- | DIQMTQSPSAMSASVGDRVTITCRASQGISHYL | 11 |
| VHH/OAH1 | VWFQQKPGKVPKRLIYAASSLQSGVPSRFSGSG | |
| LC | SGTEFTLTISSLQPEDFATYYCLQHNSYPWTFG | |
| | QGTKVEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| Fab-VHH2 | EVQLVESGGGLVKPGGSLRLSCVASGFTFSSYS | 12 |
| HC | MNWVRQAPGKGLEWVSSISSSSSYIYYADSVKG | |
| | RFTISRDNAKNSLYLQMNSLRAEDTAVYYCARR | |
| | HGYSNSDAFDNWGQGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLG | |
| | TQTYICNVNHKPSNTKVDKRVEPKSCGGGGQGG | |
| | GGQGGGGQGGGGQGGGGQEVQLLESGGGLVQPG | |
| | GSLRLSCAASGRYIDETAVAWFRQAPGKGREFV | |
| | AGIGGGVDITYYADSVKGRFTISRDNSKNTLYL | |
| | QMNSLRPEDTAVYYCGARPGRPLITSKVADLYP | |
| | YWGQGTLVTVSSPP | |
| OAH1 (one | EVQLVESGGGLVKPGGSLRLSCVASGFTFSSYS | 13 |
| arm | MNWVRQAPGKGLEWVSSISSSSSYIYYADSVKG | |
| heteromab) | RFTISRDNAKNSLYLQMNSLRAEDTAVYYCARR | |
| HC1 | HGYSNSDAFDNWGQGTLVTVSSASTKGPCVFPL | |
| | APCSRSTSESTAALGCLVKDYFPEPVTVSWNSG | |
| | ALTSGVHTFPAVLQSSGLYSLSSVVTVPSSSLG | |
| | TKTYTCNVDHKPSNTKVDKRVESKYGPPCPPCP | |
| | APEAAGGPSVFLFPPKPKDTLMISRTPEVTCVV | |
| | VDVSQEDPEVQFNWYVDGVEVHNAKTKPREEQF | |
| | NSTYRVVSVLTVLHQDWLNGKEYKCKVSNKGLP | |
| | SSIEKTISKAKGQPREPQVSTLPPSQEEMTKNQ | |
| | VSLMCLVYGFYPSDIAVEWESNGQPENNYKTTP | |
| | PVLDSDGSFFLYSVLTVDKSRWQEGNVFSCSVM | |
| | HEALHNHYTQKSLSLSLG | |
| OAH1 HC2 | ESKYGPPCPPCPAPEAAGGPSVFLFPPKPKDTL | 14 |
| | MISRTPEVTCVVVDVSQEDPEVQFNWYVDGVEV | |
| | HNAKTKPREEQFNSTYRVVSVLTVLHQDWLNGK | |
| | EYKCKVSNKGLPSSIEKTISKAKGQPREPQVYT | |
| | LPPSQGDMTKNQVQLTCLVKGFYPSDIAVEWES | |
| | NGQPENNYKTTPPVLDSDGSFFLASRLTVDKSR | |
| | WQEGNVFSCSVMHEALHNHYTQKSLSLSLG | |
| OAH2 (one | EVQLVESGGGLVKPGGSLRLSCVASGFTFSSYS | 15 |
| arm | MNWVRQAPGKGLEWVSSISSSSSYIYYADSVKG | |
| heteromab) | RFTISRDNAKNSLYLQMNSLRAEDTAVYYCARR | |
| HC1 | HGYSNSDAFDNWGQGTLVTVSSASTKGPSVFPL | |
| | APVSRSTSESTAALGCLVKDYFPEPVTVSWNSG | |
| | ALTSGVHTFPAVLQSSGLYSLSSVVTVPSSSLG | |
| | TKTYTCNVDHKPSNTKVDKRVESKYGPPCPPVP | |
| | APEAAGGPSVFLFPPKPKDTLMISRTPEVTCVV | |
| | VDVSQEDPEVQFNWYVDGVEVHNAKTKPREEQF | |
| | NSTYRVVSVLTVLHQDWLNGKEYKCKVSNKGLP | |
| | SSIEKTISKAKGQPREPQVSTLPPSQEEMTKNQ | |
| | VSLMCLVYGFYPSDIAVEWESNGQPENNYKTTP | |
| | PVLDSDGSFFLYSVLTVDKSRWQEGNVFSCSVM | |
| | HEALHNHYTQKSLSLSLG | |
| OAH2 HC2 | ESKYGPPCPPVPAPEAAGGPSVFLFPPKPKDTL | 16 |
| | MISRTPEVTCVVVDVSQEDPEVQFNWYVDGVEV | |
| | HNAKTKPREEQFNSTYRVVSVLTVLHQDWLNGK | |
| | EYKCKVSNKGLPSSIEKTISKAKGQPREPQVYT | |
| | LPPSQGDMTKNQVQLTCLVKGFYPSDIAVEWES | |
| | NGQPENNYKTTPPVLDSDGSFFLASRLTVDKSR | |
| | WQEGNVFSCSVMHEALHNHYTQKSLSLSLG | |
| OAH2 LC | DIQMTQSPSAMSASVGDRVTITCRASQGISHYL | 17 |
| | VWFQQKPGKVPKRLIYAASSLQSGVPSRFSGSG | |
| | SGTEFTLTISSLQPEDFATYYCLQHNSYPWTFG | |
| | QGTKVEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEV | |

TABLE 1b
Exemplary sequences of human TfR binding
antibody or antibody fragment

| | HC1 | LC1 | HC2 | LC2 |
|---|---|---|---|---|
| A1 | SEQ ID NO: | SEQ ID NO: | N/A | N/A |
| (Fab) | 9 | 11 | | |
| A2 | SEQ ID NO: | SEQ ID NO: | N/A | N/A |
| (Fab-VHH1) | 10 | 11 | | |
| A3 | SEQ ID NO: | SEQ ID NO: | N/A | N/A |
| (Fab-VHH2) | 12 | 11 | | |
| A4 | SEQ ID NO: | SEQ ID NO: | SEQ ID NO: | N/A |
| (One Arm | 13 | 11 | 14 | |
| Heteromab 1) | | | | |
| A5 | SEQ ID NO: | SEQ ID NO: | SEQ ID NO: | N/A |
| (One Arm | 15 | 17 | 16 | |
| Heteromab 2) | | | | |

In some embodiments, the antibody or antibody fragment is a monoclonal antibody.

In some embodiments, the antibody or antibody fragment is a Fab.

In some embodiments, the antibody or antibody fragment further comprises a half-life extender, e.g., a VHH that binds human serum albumin (HSA). In some embodiments, the antibody or antibody fragment is a Fab-VHH. In some embodiments, the VHH also binds mouse, rat, and/or cynomolgus monkey albumin. An exemplary VHH that binds human HSA is shown in Table 2. In some embodiments, such a VHH comprises CDR1 comprising SEQ ID NO: 18, CDR2 comprising SEQ ID NO: 19, and CDR3 comprising SEQ ID NO: 20. In some embodiments, such a VHH comprises SEQ ID NO: 21. In some embodiments, the VHH is linked to the TfR binding domain through a peptide linker, e.g., (GGGGQ)₄ (SEQ ID NO: 22). In some embodiments, the VHH is linked to the C-terminus of the TfR binding domain.

TABLE 2
Exemplary sequences of VHH
that binds human serum albumin (HSA)

| | | SEQ |
|---|---|---|
| | | ID |
| Region | Sequence | NO |
| CDR1 | ETAVA | 18 |
| (KABAT) | | |
| CDR2 | GIGGGVDITYYADSVKG | 19 |
| (KABAT) | | |
| CDR3 | RPGRPLITSKVADLYPY | 20 |
| (KABAT) | | |
| VHH full | EVQLLESGGGLVQPGGSLRLSCAASGRYIDE | 21 |
| length | TAVAWFRQAPGKGREFVAGIGGGVDITYYAD | |
| | SVKGRFTISRDNSKNTLYLQMNSLRPEDTAV | |
| | YYCGARPGRPLITSKVADLYPYWGQGTLVTV | |
| | SSPP | |
| Optional | GGGGQGGGGQGGGGQGGGGQ | 22 |
| linker | | |

In some embodiments, the antibody or antibody fragment is heterodimeric antibody. Heterodimeric antibodies such as heteromab, orthomab or duobody have been described in WO2014150973, WO2016118742, WO2018118616, and WO2011131746.

In some embodiments, the antibody or antibody fragment comprises heterodimeric mutations. In some embodiments, the antibody or antibody fragment comprises a modified Fc region comprising a first Fc CH3 domain comprising serine at residue 349, methionine at residue 366, tyrosine at residue 370, and valine at residue 409, and a second Fc CH3 domain comprising glycine at residue 356, aspartic acid at residue 357, glutamine at residue 364 and alanine at residue 407 (all residues are numbered according to the EU Index numbering). In some embodiments, the antibody or antibody fragment comprises a modified Fc region comprising a first Fc CH3 domain comprising leucine at residue 405, and a second Fc CH3 domain comprising arginine at residue 409 (all residues are numbered according to the EU Index numbering).

In some embodiments, the antibody or antibody fragment comprises two or more native cysteine residues, which can be used for conjugation. For example, in some embodiments, the antibody or antibody fragment comprises a native cysteine at position 220 of the light chain and a native cysteine at position 226 of the heavy chain, which can be used for conjugation (all residues according to the EU Index numbering).

In some embodiments, the antibody or antibody fragment comprises engineered cysteine residues for conjugation. The approach of including engineered cysteines as a means for conjugation has been described in WO 2018/232088. In some embodiments, the antibody or antibody fragment comprises a heavy chain comprising one or more cysteines at the following residues: 124, 157, 162, 262, 373, 375, 378, 397, 415 (all residues according to the EU Index numbering). In some embodiments, the antibody or antibody fragment comprises a light chain (e.g., a kappa light chain) comprising one or more cysteines at the following residues: 156, 171, 191, 193, 202, 208 (all residues according to the EU Index numbering). In some embodiments, the antibody or antibody fragment comprises a heavy chain constant region comprising cysteine at residue 124 (according to the EU Index numbering). In some embodiments, the antibody or antibody fragment comprises a light chain constant region comprising cysteine at residue 156 (according to the EU Index numbering). In some embodiments, the antibody or antibody fragment comprises an immunoglobulin Fc region comprising cysteine at residue 378 (according to the EU Index numbering).

In some embodiments, the antibody or antibody fragment is any one of the human TfR binding antibody or antibody fragments in Table 1b.

In some embodiments, the human TfR binding antibody or antibody fragment has a Fab format, e.g., A1. In some embodiments, the human TfR binding antibody or antibody fragment comprises one HC and one LC, and wherein the HC comprises SEQ ID NO: 9 and the LC comprises SEQ ID NO: 10.

In some embodiments, the human TfR binding antibody or antibody fragment has a Fab-VHH format, e.g., A2 or A3. In some embodiments, the human TfR antibody or antibody fragments comprises one HC and one LC, wherein the HC comprises SEQ ID NO: 10 and the LC comprises SEQ ID NO: 11. In some embodiments, the human TfR antibody or antibody fragments comprises one HC and one LC, wherein the HC comprises SEQ ID NO: 12 and the LC comprises SEQ ID NO: 11.

In some embodiments, the human TfR binding antibody or antibody fragment has a one arm heteromab format, e.g., A4 or A5. In some embodiments, the human TfR binding antibody or antibody fragment comprises two heavy chains HC1 and HC2 and one light chain LC1, wherein HC1 comprises SEQ ID NO: 13, LC1 comprises SEQ ID NO: 11, HC2 comprises SEQ ID NO: 14. In some embodiments, the human TfR binding antibody or antibody fragment comprises two heavy chains HC1 and HC2 and one light chain LC1, wherein HC1 comprises SEQ ID NO: 15, LC1 comprises SEQ ID NO: 17, HC2 comprises SEQ ID NO: 16.

The antibody or antibody fragments described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the antibody or antibody fragment) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of the antibody or antibody fragments described herein. According to some embodiments, a host cell may be stably or transiently transfected, transformed, transduced or infected with an expression vector expressing HC polypeptides and an expression vector expressing LC polypeptides of the antibody or antibody fragments described herein. In some embodiments, a host cell may be stably or transiently transfected, transformed, transduced or infected with an expression vector expressing HC and LC polypeptides of the antibody or antibody fragments described herein. The antibody or antibody fragments may be produced in mammalian cells such as CHO, NS0, HEK293 or COS cells according to techniques well known in the art.

Medium, into which the antibody or antibody fragments has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

### Therapeutic Agent

In some embodiments, the therapeutic agent (D) is an oligonucleotide (e.g., antisense oligonucleotide), double stranded RNA (e.g., siRNA, saRNA), polypeptide, small molecule, nanoparticle, lipid nanoparticle, exosome, or a combination thereof. In some embodiments, the therapeutic agent is an oligonucleotide. In some embodiments, the therapeutic agent is a double stranded RNA (dsRNA).

In some embodiments, the therapeutic agent is a double stranded RNA (dsRNA) comprising a sense stand and an antisense strand. In some embodiments, the dsRNA comprises an antisense strand complementary to a target mRNA selected from SNCA, MAPT, APP, ATXN2, ATXN3, SARM1, APOE, BACE1, FMR1, LRRK2, HTT, SOD1, SCN10A, SCN9A, CACNA1B, or PRNP mRNA. In some embodiments, the dsRNA comprises an antisense strand complementary to SNCA mRNA.

In some embodiments, the sense strand and the antisense strand of the dsRNA are each 15-30 nucleotides in length, e.g., 20-25 nucleotides in length. In some embodiments, the dsRNA has a sense strand of 21 nucleotides and an antisense strand of 23 nucleotides. In some embodiments, the sense strand and antisense strand of the dsRNA may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

Exemplary unmodified sense strand and antisense strand sequences of dsRNA targeting human SNCA mRNA are provided in Table 3a.

TABLE 3a
Unmodified Nucleic Acid Sequences of dsRNA
targeting human SNCA mRNA (SNCA siRNA)

| | | | | | Start |
|---|---|---|---|---|---|
| | | | | | position |
| | | | | | of target |
| | | | | | region on |
| | | | | | human |
| | Sense | SEQ | Antisense | SEQ | SNCA |
| dsRNA | Strand | ID | Strand | ID | transcript |
| No. | (5′ to 3′) | NO | (5′ to 3′) | NO | NM_000345.4 |
| 1 | CUGUACAAGU | 23 | UGGAACUGAG | 24 | 701 |
| | GCUCAGUUCC | | CACUUGUACA | | |
| | A | | GGA | | |
| 2 | UUGUACAAGU | 25 | UGGAACUGAG | 24 | 701 |
| | GCUCAGUUCC | | CACUUGUACA | | |
| | A | | GGA | | |
| 3 | UGUACAAGUG | 26 | UUGGAACUGA | 27 | 702 |
| | CUCAGUUCCA | | GCACUUGUAC | | |
| | A | | AGG | | |

In some embodiments, the dsRNA targets SNCA mRNA. In some embodiments, the sense strand and the antisense strand of the dsRNA comprise a pair of nucleic acid sequences selected from the group consisting of:
(a) the sense strand comprises a first nucleic acid sequence having at least 90% sequence identity to SEQ ID NO: 23, and the antisense strand comprises a second nucleic acid sequence having at least 90% sequence identity to SEQ ID NO: 24;
(b) the sense strand comprises a first nucleic acid sequence having at least 90% sequence identity to SEQ ID NO: 25, and the antisense strand comprises a second nucleic acid sequence having at least 90% sequence identity to SEQ ID NO: 24; and
(c) the sense strand comprises a first nucleic acid sequence having at least 90% sequence identity to SEQ ID NO: 26, and the antisense strand comprises a second nucleic acid sequence having at least 90% sequence identity to SEQ ID NO: 27,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises a first nucleic acid sequence having at least 95% sequence identity to SEQ ID NO: 23, and the antisense strand comprises a second nucleic acid sequence having at least 95% sequence identity to SEQ ID NO: 24;
(b) the sense strand comprises a first nucleic acid sequence having at least 95% sequence identity to SEQ ID NO: 25, and the antisense strand comprises a second nucleic acid sequence having at least 95% sequence identity to SEQ ID NO: 24; and
(c) the sense strand comprises a first nucleic acid sequence having at least 95% sequence identity to SEQ ID NO: 26, and the antisense strand comprises a second nucleic acid sequence having at least 95% sequence identity to SEQ ID NO: 27,
   wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises SEQ ID NO: 23, and the antisense strand comprises SEQ ID NO: 24;
(b) the sense strand comprises SEQ ID NO: 25, and the antisense strand comprises SEQ ID NO: 24; and
(c) the sense strand comprises SEQ ID NO: 26, and the antisense strand comprises SEQ ID NO: 27,
   wherein optionally one or more nucleotides of the sense strand and the antisense strand are independently modified nucleotides, and wherein optionally one or more internucleotide linkages of the sense strand and the antisense strand are modified internucleotide linkages.

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl (e.g., 2′-O—C₁₆ alkyl).

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl (e.g., 2′-O—C₁₆ alkyl) modified nucleotide.

In some embodiments, the sense strand has four 2′-fluoro modified nucleotides, e.g., at positions 7, 9, 10, 11 from the 5′ end of the sense strand. In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 4.

TABLE 4
Abasic or inverted abasic (iAb) moieties

| | Structure |
|---|---|
| 1 (abasic) | 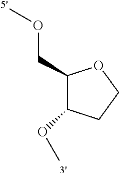 |
| 2 (iAb) | 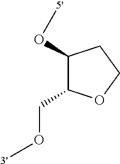 |

“5′” and “3′” indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SNCA mRNA are provided in Table 3b.

In some embodiments, the dsRNA comprises a sense strand that comprises a sequence that has 1, 2, or 3 differences from a sense stand sequence in Table 3a or 3b. In some embodiments, the dsRNA comprises an antisense strand that comprises a sequence that has 1, 2, or 3 differences from an antisense stand sequence in Table 3a or 3b.

TABLE 3b
Modified Nucleic Acid Sequences of dsRNA
targeting human SNCA mRNA (SNCA siRNA)

| | | | SEQ |
|---|---|---|---|
| dsRNA | | | ID |
| No. | Strand | Oligo Sequence 5′ to 3′ | NO |
| 4 | S | mC*mU*mGmUmAmCmAmAfGfUfG | 28 |
| | | mCmUmCmAmGmUmUmC*mC*mA* | |
| | AS | mU*fG*mGmAfAmCmUfGmAmGmC | 29 |
| | | mAmCfUmUfGmUmAmCmAmG*mG* | |
| | | mA | |
| 5 | S | [NH₂mU]*mU*mGmUmAmCmAmAf | 30 |
| | | GfUfGmCmUmCmAmGmUmUmC* | |
| | | mC*mA | |
| | AS | mU*fG*mGmAfAmCmUfGmAmGmC | 29 |
| | | mAmCfUmUfGmUmAmCmAmG*mG* | |
| | | mA | |
| 6 | S | mU*mG*mUmAmCmAmAmGfUfGfC | 31 |
| | | mUmCmAmGmUmUmCmC*mA*mA* | |
| | AS | mU*fU*mGmGfAmAmCfUmGmAmG | 32 |
| | | mCmAfCmUfUmGmUmAmCmA*mG* | |
| | | mG | |

Note-
The 5′ end of the AS may be substituted with 5′-vinylphosphonate.
Abbreviations-
“m” indicates 2′-OMe;
“f” indicated 2′-fluoro;
“*” indicates phosphorothioate linkage;
“VP” indicates 5′-vinylphosphonate;
“NH₂” indicates a 5′-amino group;
“S” means the sense strand;
“AS” means the antisense strand.

In some embodiments, the dsRNA targets SNCA mRNA. In some embodiments, the sense strand and the antisense strand of the dsRNA comprise a pair of nucleic acid sequences selected from the group consisting of:
(a) the sense strand comprises SEQ ID NO: 28, and the antisense strand comprises SEQ ID NO: 29;
(b) the sense strand comprises SEQ ID NO: 30, and the antisense strand comprises SEQ ID NO: 29; and
(c) the sense strand comprises SEQ ID NO:31, and the antisense strand comprises 32.

In some embodiments, the sense strand and the antisense strand of the dsRNA have a pair of nucleic acid sequences selected from the group consisting of:
(a) the sense strand consists of SEQ ID NO: 28, and the antisense strand consists of SEQ ID NO: 29;
(b) the sense strand consists of SEQ ID NO: 30, and the antisense strand consists of SEQ ID NO: 29; and
(c) the sense strand consists of SEQ ID NO: 31, and the antisense strand consists of SEQ ID NO: 32.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene)amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

### Pharmaceutical Composition

In another aspect, provided herein are pharmaceutical compositions comprising any of the conjugates described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating a neurodegenerative disease, in a patient in need thereof, and such the method comprises administering to the patient an effective amount of the conjugate or a pharmaceutical composition described herein.

In a further aspect, provided herein are methods of treating a neurodegenerative disease in a patient in need thereof, and such the method comprises administering to the patient an effective amount of the conjugate or a pharmaceutical composition described herein. Exemplary neurodegenerative disease includes, but are not limited to, Parkinson's disease; multiple system atrophy; Lewy body dementia or dementia with Lewy bodies; pure autonomic failure; Alzheimer's disease; Lewy body dysphagia; and incidental Lewy body disease. In some embodiments, the neurodegenerative disease is Parkinson's disease, Alzheimer's disease, multiple system atrophy, or Lewy body dementia. The conjugate or a pharmaceutical composition can be administered to the patient intravenously or subcutaneously.

The conjugate dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are conjugates described herein or pharmaceutical compositions comprising such conjugates for use in a therapy. Also provided herein are conjugates described herein or pharmaceutical compositions comprising such conjugates for use in the treatment of a neurodegenerative disease.

### Method of Making

In another aspect, provided herein are methods of generating a conjugate, such methods comprising reacting an antibody comprising two cysteine residues with a compound comprising any one of the following formula:

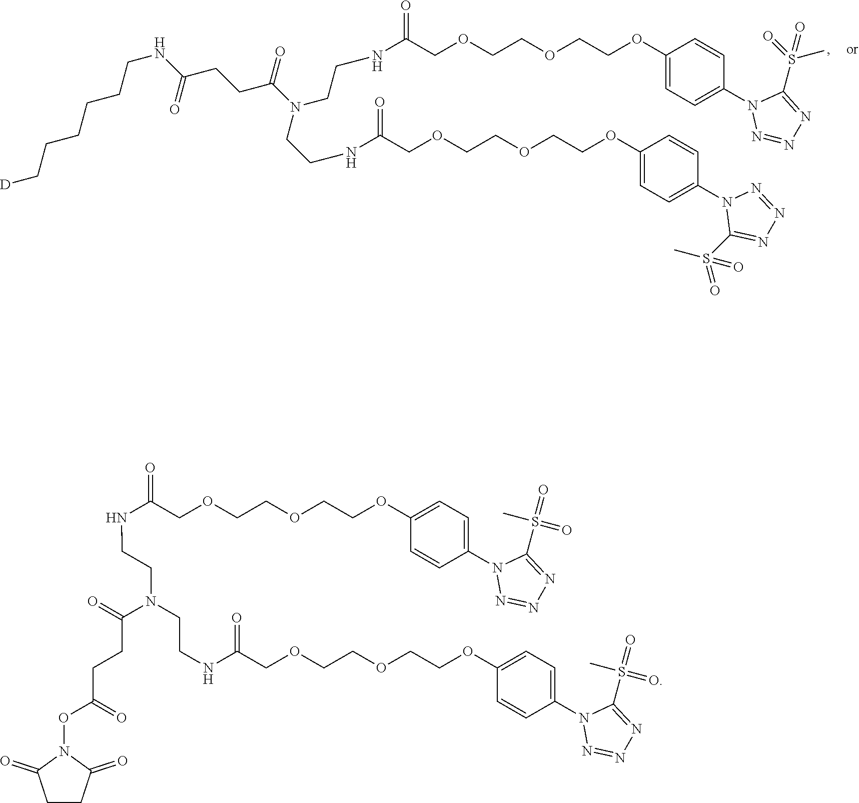 [SMILES: [2H]CCCCCCNC(=O)CCC(=O)N(CCNC(=O)COCCOCCOc1ccc(-n2nnnc2S(C)(=O)=O)cc1)CCNC(=O)COCCOCCOc1ccc(-n2nnnc2S(C)(=O)=O)cc1 ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “heterodimeric antibody”, as used herein, refers to an antibody that comprises two distinct antigen-binding domains.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a compound, protein or conjugate may vary according to factors such as the disease state, age, sex, and weight of the individual, and the ability of the compound, protein or conjugate to elicit a desired response in the individual. An effective amount is also one in which any toxic or detrimental effects of the compound, protein or conjugate are outweighed by the therapeutically beneficial effects.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 4.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length. An oligonucleotide can be single stranded or double stranded.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “polypeptide” or “protein”, as used herein, refers to a polymer of amino acid residues. The term applies to polymers comprising naturally occurring amino acids and polymers comprising one or more non-naturally occurring amino acids.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “SNCA” refers to an alpha-synuclein (SNCA) mRNA, protein, or polypeptide. The nucleic acid sequence of a human SNCA mRNA transcript can be found at NM 000345.4:

| (SEQ ID NO: 33) | |
|---|---|
| 1 | GGCGACGACC AGAAGGGGCC CAAGAGAGGG GGCGAGCGAC CGAGCGCCGC GACGCGGAAG |
| 61 | TGAGGTGCGT GCGGGCTGCA GCGCAGACCC CGGCCCGGCC CCTCCGAGAG CGTCCTGGGC |
| 121 | GCTCCCTCAC GCCTTGCCTT CAAGCCTTCT GCCTTTCCAC CCTCGTGAGC GGAGAACTGG |
| 181 | GAGTGGCCAT TCGACGACAG TGTGGTGTAA AGGAATTCAT TAGCCATGGA TGTATTCATG |
| 241 | AAAGGACTTT CAAAGGCCAA GGAGGGAGTT GTGGCTGCTG CTGAGAAAAC CAAACAGGGT |
| 301 | GTGGCAGAAG CAGCAGGAAA GACAAAAGAG GGTGTTCTCT ATGTAGGCTC CAAAACCAAG |
| 361 | GAGGGAGTGG TGCATGGTGT GGCAACAGTG GCTGAGAAGA CCAAAGAGCA AGTGACAAAT |
| 421 | GTTGGAGGAG CAGTGGTGAC GGGTGTGACA GCAGTAGCCC AGAAGACAGT GGAGGGAGCA |
| 481 | GGGAGCATTG CAGCAGCCAC TGGCTTTGTC AAAAAGGACC AGTTGGGCAA GAATGAAGAA |
| 541 | GGAGCCCCAC AGGAAGGAAT TCTGGAAGAT ATGCCTGTGG ATCCTGACAA TGAGGCTTAT |
| 601 | GAAATGCCTT CTGAGGAAGG GTATCAAGAC TACGAACCTG AAGCCTAAGA AATATCTTTG |
| 661 | CTCCCAGTTT CTTGAGATCT GCTGACAGAT GTTCCATCCT GTACAAGTGC TCAGTTCCAA |
| 721 | TGTGCCCAGT CATGACATTT CTCAAAGTTT TTACAGTGTA TCTCGAAGTC TTCCATCAGC |
| 781 | AGTGATTGAA GTATCTGTAC CTGCCCCCAC TCAGCATTTC GGTGCTTCCC TTTCACTGAA |
| 841 | GTGAATACAT GGTAGCAGGG TCTTTGTGTG CTGTGGATTT TGTGGCTTCA ATCTACGATG |
| 901 | TTAAAACAAA TTAAAAACAC CTAAGTGACT ACCACTTATT TCTAAATCCT CACTATTTTT |
| 961 | TTGTTGCTGT TGTTCAGAAG TTGTTAGTGA TTTGCTATCA TATATTATAA GATTTTTAGG |
| 1021 | TGTCTTTTAA TGATACTGTC TAAGAATAAT GACGTATTGT GAAATTTGTT AATATATATA |
| 1081 | ATACTTAAAA ATATGTGAGC ATGAAACTAT GCACCTATAA ATACTAAATA TGAAATTTTA |
| 1141 | CCATTTTGCG ATGTGTTTTA TTCACTTGTG TTTGTATATA AATGGTGAGA ATTAAAATAA |
| 1201 | AACGTTATCT CATTGCAAAA ATATTTTATT TTTATCCCAT CTCACTTTAA TAATAAAAAT |
| 1261 | CATGCTTATA AGCAACATGA ATTAAGAACT GACACAAAGG ACAAAAATAT AAAGTTATTA |
| 1321 | ATAGCCATTT GAAGAAGGAG GAATTTTAGA AGAGGTAGAG AAAATGGAAC ATTAACCCTA |
| 1381 | CACTCGGAAT TCCCTGAAGC AACACTGCCA GAAGTGTGTT TTGGTATGCA CTGGTTCCTT |
| 1441 | AAGTGGCTGT GATTAATTAT TGAAAGTGGG GTGTTGAAGA CCCCAACTAC TATTGTAGAG |
| 1501 | TGGTCTATTT CTCCCTTCAA TCCTGTCAAT GTTTGCTTTA CGTATTTTGG GGAACTGTTG |
| 1561 | TTTGATGTGT ATGTGTTTAT AATTGTTATA CATTTTTAAT TGAGCCTTTT ATTAACATAT |
| 1621 | ATTGTTATTT TTGTCTCGAA ATAATTTTTT AGTTAAAATC TATTTTGTCT GATATTGGTG |
| 1681 | TGAATGCTGT ACCTTTCTGA CAATAAATAA TATTCGACCA TGAATAAAAA AAAAAAAAAA |
| 1741 | GTGGGTTCCC GGGAACTAAG CAGTGTAGAA GATGATTTTG ACTACACCCT CCTTAGAGAG |
| 1801 | CCATAAGACA CATTAGCACA TATTAGCACA TTCAAGGCTC TGAGAGAATG TGGTTAACTT |
| 1861 | TGTTTAACTC AGCATTCCTC ACTTTTTTTT TTTAATCATC AGAAATTCTC TCTCTCTCTC |
| 1921 | TCTCTTTTTC TCTCGCTCTC TTTTTTTTTT TTTTTTTACA GGAAATGCCT TTAAACATCG |
| 1981 | TTGGAACTAC CAGAGTCACC TTAAAGGAGA TCAATTCTCT AGACTGATAA AAATTTCATG |
| 2041 | GCCTCCTTTA AATGTTGCCA AATATATGAA TTCTAGGATT TTTCCTTAGG AAAGGTTTTT |
| 2101 | CTCTTTCAGG GAAGATCTAT TAACTCCCCA TGGGTGCTGA AAATAAACTT GATGGTGAAA |
| 2161 | AACTCTGTAT AAATTAATTT AAAAATTATT TGGTTTCTCT TTTTAATTAT TCTGGGGCAT |
| 2221 | AGTCATTTCT AAAAGTCACT AGTAGAAAGT ATAATTTCAA GACAGAATAT TCTAGACATG |
| 2281 | CTAGCAGTTT ATATGTATTC ATGAGTAATG TGATATATAT TGGGCGCTGG TGAGGAAGGA |
| 2341 | AGGAGGAATG AGTGACTATA AGGATGGTTA CCATAGAAAC TTCCTTTTTT ACCTAATTGA |
| 2401 | AGAGAGACTA CTACAGAGTG CTAAGCTGCA TGTGTCATCT TACACTAGAG AGAAATGGTA |
| 2461 | AGTTTCTTGT TTTATTTAAG TTATGTTTAA GCAAGGAAAG GATTTGTTAT TGAACAGTAT |
| 2521 | ATTTCAGGAA GGTTAGAAAG TGGCGGTTAG GATATATTTT AAATCTACCT AAAGCAGCAT |
| 2581 | ATTTTAAAAA TTTAAAAGTA TTGGTATTAA ATTAAGAAAT AGAGGACAGA ACTAGACTGA |
| 2641 | TAGCAGTGAC CTAGAACAAT TTGAGATTAG GAAAGTTGTG ACCATGAATT TAAGGATTTA |
| 2701 | TGTGGATACA AATTCTCCTT TAAAGTGTTT CTTCCCTTAA TATTTATCTG ACGGTAATTT |
| 2761 | TTGAGCAGTG AATTACTTTA TATATCTTAA TAGTTTATTT GGGACCAAAC ACTTAAACAA |
| 2821 | AAAGTTCTTT AAGTCATATA AGCCTTTTCA GGAAGCTTGT CTCATATTCA CTCCCGAGAC |
| 2881 | ATTCACCTGC CAAGTGGCCT GAGGATCAAT CCAGTCCTAG GTTTATTTTG CAGACTTACA |
| 2941 | TTCTCCCAAG TTATTCAGCC TCATATGACT CCACGGTCGG CTTTACCAAA ACAGTTCAGA |
| 3001 | GTGCACTTTG GCACACAATT GGGAACAGAA CAATCTAATG TGTGGTTTGG TATTCCAAGT |
| 3061 | GGGGTCTTTT TCAGAATCTC TGCACTAGTG TGAGATGCAA ACATGTTTCC TCATCTTTCT |
| 3121 | GGCTTATCCA GTATGTAGCT ATTTGTGACA TAATAAATAT ATACATATAT GAAAATA. |

The amino acid sequence of a human SNCA protein can be found at NP_000336.1:

| (SEQ ID NO: 34) | |
|---|---|
| 1 | MDVFMKGLSK AKEGVVAAAE KTKQGVAEAA GKTKEGVLYV GSKTKEGVVH GVATVAEKTK |
| 61 | EQVTNVGGAV VTGVTAVAQK TVEGAGSIAA ATGFVKKDQL GKNEEGAPQE GILEDMPVDP |
| 121 | DNEAYEMPSE EGYQDYEPEA. |

The nucleic acid sequence of a mouse SNCA mRNA transcript can be found at NM_001042451.2; and the amino acid sequence of a mouse SNCA protein can be found at NP_001035916.1. The nucleic acid sequence of a rat SNCA mRNA transcript can be found at NM_019169.3; and the amino acid sequence of a rat SNCA protein can be found at NP_062042.1. The nucleic acid sequence of a monkey SNCA mRNA transcript can be found at XM_005555422.2; and the amino acid sequence of a monkey SNCA protein can be found at XP_005555479.1.

As used herein, “TfR” refers to a transferrin receptor protein or polypeptide, e.g., a human or mouse transferrin receptor protein or polypeptide. The amino acid sequence of the human transferrin receptor protein (hTFR) can be found at NP_001121620.1:

| (SEQ ID NO: 35) | |
|---|---|
| 1 | MMDQARSAFS NLFGGEPLSY TRFSLARQVD GDNSHVEMKL AVDEEENADN NTKANVTKPK |
| 61 | RCSGSICYGT IAVIVFFLIG FMIGYLGYCK GVEPKTECER LAGTESPVRE EPGEDFPAAR |
| 121 | RLYWDDLKRK LSEKLDSTDF TGTIKLLNEN SYVPREAGSQ KDENLALYVE NQFREFKLSK |
| 181 | VWRDQHFVKI QVKDSAQNSV IIVDKNGRLV YLVENPGGYV AYSKAATVTG KLVHANFGTK |
| 241 | KDFEDLYTPV NGSIVIVRAG KITFAEKVAN AESLNAIGVL IYMDQTKFPI VNAELSFFGH |
| 301 | AHLGTGDPYT PGFPSFNHTQ FPPSRSSGLP NIPVQTISRA AAEKLFGNME GDCPSDWKTD |
| 361 | STCRMVTSES KNVKLTVSNV LKEIKILNIF GVIKGFVEPD HYVVVGAQRD AWGPGAAKSG |
| 421 | VGTALLLKLA QMFSDMVLKD GFQPSRSIIF ASWSAGDFGS VGATEWLEGY LSSLHLKAFT |
| 481 | YINLDKAVLG TSNFKVSASP LLYTLIEKTM QNVKHPVTGQ FLYQDSNWAS KVEKLTLDNA |
| 541 | AFPFLAYSGI PAVSFCFCED TDYPYLGTTM DTYKELIERI PELNKVARAA AEVAGQFVIK |
| 601 | LTHDVELNLD YERYNSQLLS FVRDLNQYRA DIKEMGLSLQ WLYSARGDFF RATSRLTTDF |
| 661 | GNAEKTDRFV MKKLNDRVMR VEYHFLSPYV SPKESPFRHV FWGSGSHTLP ALLENLKLRK |
| 721 | QNNGAFNETL FRNQLALATW TIQGAANALS GDVWDIDNEF. |

The amino acid sequence of the mouse transferrin receptor protein (mTFR) can be found at NP_001344227.1:

| (SEQ ID NO: 36) | |
|---|---|
| 1 | MMDQARSAFS NLFGGEPLSY TRFSLARQVD GDNSHVEMKL AADEEENADN NMKASVRKPK |
| 61 | RFNGRLCFAA IALVIFFLIG FMSGYLGYCK RVEQKEECVK LAETEETDKS ETMETEDVPT |
| 121 | SSRLYWADLK TLLSEKLNSI EFADTIKQLS QNTYTPREAG SQKDESLAYY IENQFHEFKF |
| 181 | SKVWRDEHYV KIQVKSSIGQ NMVTIVQSNG NLDPVESPEG YVAFSKPTEV SGKLVHANFG |
| 241 | TKKDFEELSY SVNGSLVIVR AGEITFAEKV ANAQSFNAIG VLIYMDKNKF PVVEADLALF |
| 301 | GHAHLGTGDP YTPGFPSFNH TQFPPSQSSG LPNIPVQTIS RAAAEKLFGK MEGSCPARWN |
| 361 | IDSSCKLELS QNQNVKLIVK NVLKERRILN IFGVIKGYEE PDRYVVVGAQ RDALGAGVAA |
| 421 | KSSVGTGLLL KLAQVFSDMI SKDGFRPSRS IIFASWTAGD FGAVGATEWL EGYLSSLHLK |
| 481 | AFTYINLDKV VLGTSNFKVS ASPLLYTLMG KIMQDVKHPV DGKSLYRDSN WISKVEKLSF |
| 541 | DNAAYPFLAY SGIPAVSFCF CEDADYPYLG TRLDTYEALT QKVPQLNQMV RTAAEVAGQL |
| 601 | IIKLTHDVEL NLDYEMYNSK LLSFMKDLNQ FKTDIRDMGL SLQWLYSARG DYFRATSRLT |
| 661 | TDFHNAEKTN RFVMREINDR IMKVEYHFLS PYVSPRESPF RHIFWGSGSH TLSALVENLK |
| 721 | LRQKNITAFN ETLFRNQLAL ATWTIQGVAN ALSGDIWNID NEF. |

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The following examples are offered to illustrate, but not to limit, the claimed inventions.

### EXAMPLES

### Example 1: Generation and Characterization of TfR Binding Antibody or Antibody Fragments

### Generation of Human or Mouse TfR Binding Antibody or Antibody Fragments

Antibody against mouse TfR was generated by immunizing New Zealand White rabbits with the extracellular domain (ECD) of mouse Transferrin Receptor 1 protein with a His tag (mTfR-ECD-6His, SEQ ID NO: 37, see Table 5). mTfR antigen positive B-cells were sorted from peripheral blood and binding of individual antibodies cloned from those B-cells was verified on his-tagged mTfR.

Antibody against human TfR was generated by immunizing AlivaMab® transgenic mice with the extracellular domains of human Transferrin Receptor 1 protein with a His tag (hTfR-ECD-6His, SEQ ID NO: 38, see Table 5) and mouse Transferrin Receptor protein (mTfR, SEQ ID NO: 36). Antigen positive B-cells were sorted from pooled spleens. Binding of individual antibodies cloned from those B-cells to his-tagged hTfR-ECD was verified.

Additional antibody against human TfR was generated by immunizing AlivaMab® transgenic mice with the apical domain of human Transferrin Receptor 1 protein with a His tag (hTfR-ApD-6His, SEQ ID NO: 39, see Table 5). Antigen positive B-cells were sorted from pooled spleens. Binding of individual antibodies cloned from those B-cells to his-tagged hTfR-ECD was verified.

TABLE 5
Sequences of the immunogens used to
generate human or mouse TfR antibodies.

| | | SEQ |
|---|---|---|
| | | ID |
| Immunogen | Sequence | NO |
| mTfR-ECD- | HHHHHHCKRVEQKEECVKLAETEETDKSETMET | 37 |
| 6His | EDVPTSSRLYWADLKTLLSEKLNSIEFADTIKQ | |
| | LSQNTYTPREAGSQKDESLAYYIENQFHEFKFS | |
| | KVWRDEHYVKIQVKSSIGQNMVTIVQSNGNLDP | |
| | VESPEGYVAFSKPTEVSGKLVHANFGTKKDFEE | |
| | LSYSVNGSLVIVRAGEITFAEKVANAQSFNAIG | |
| | VLIYMDKNKFPVVEADLALFGHAHLGTGDPYTP | |
| | GFPSFNHTQFPPSQSSGLPNIPVQTISRAAAEK | |
| | LFGKMEGSCPARWNIDSSCKLELSQNQNVKLIV | |
| | KNVLKERRILNIFGVIKGYEEPDRYVVVGAQRD | |
| | ALGAGVAAKSSVGTGLLLKLAQVFSDMISKDGF | |
| | RPSRSIIFASWTAGDFGAVGATEWLEGYLSSLH | |
| | LKAFTYINLDKVVLGTSNFKVSASPLLYTLMGK | |
| | IMQDVKHPVDGKSLYRDSNWISKVEKLSFDNAA | |
| | YPFLAYSGIPAVSFCFCEDADYPYLGTRLDTYE | |
| | ALTQKVPQLNQMVRTAAEVAGQLIIKLTHDVEL | |
| | NLDYEMYNSKLLSFMKDLNQFKTDIRDMGLSLQ | |
| | WLYSARGDYFRATSRLTTDFHNAEKTNRFVMRE | |
| | INDRIMKVEYHFLSPYVSPRESPFRHIFWGSGS | |
| | HTLSALVENLKLRQKNITAFNETLFRNQLALAT | |
| | WTIQGVANALSGDIWNIDNEF | |
| hTfR-ECD- | HHHHHHCKGVEPKTECERLAGTESPVREEPGED | 38 |
| 6His | FPAARRLYWDDLKRKLSEKLDSTDFTGTIKLLN | |
| | ENSYVPREAGSQKDENLALYVENQFREFKLSKV | |
| | WRDQHFVKIQVKDSAQNSVIIVDKNGRLVYLVE | |
| | NPGGYVAYSKAATVTGKLVHANFGTKKDFEDLY | |
| | TPVNGSIVIVRAGKITFAEKVANAESLNAIGVL | |
| | IYMDQTKFPIVNAELSFFGHAHLGTGDPYTPGF | |
| | PSFNHTQFPPSRSSGLPNIPVQTISRAAAEKLF | |
| | GNMEGDCPSDWKTDSTCRMVTSESKNVKLTVSN | |
| | VLKEIKILNIFGVIKGFVEPDHYVVVGAQRDAW | |
| | GPGAAKSGVGTALLLKLAQMFSDMVLKDGFQPS | |
| | RSIIFASWSAGDFGSVGATEWLEGYLSSLHLKA | |
| | FTYINLDKAVLGTSNFKVSASPLLYTLIEKTMQ | |
| | NVKHPVTGQFLYQDSNWASKVEKLTLDNAAFPF | |
| | LAYSGIPAVSFCFCEDTDYPYLGTTMDTYKELI | |
| | ERIPELNKVARAAAEVAGQFVIKLTHDVELNLD | |
| | YERYNSQLLSFVRDLNQYRADIKEMGLSLQWLY | |
| | SARGDFFRATSRLTTDFGNAEKTDRFVMKKLND | |
| | RVMRVEYHFLSPYVSPKESPFRHVFWGSGSHTL | |
| | PALLENLKLRKQNNGAFNETLFRNQLALATWTI | |
| | QGAANALSGDVWDIDNEF | |
| hTfR-ApD- | HHHHHHHHGKPIPNPLLGLDSTGGGGSDSAQNS | 39 |
| 6His | VIIVDKNGRLVYLVENPGGYVAYSKAATVTGKL | |
| | VHANFGTKKDFEDLYTPVNGSIVIVRAGKITFA | |
| | EKVANAESLNAIGVLIYMDQTKFPIVNAELSFF | |
| | GHAHLGGGGGGLPNIPVQTISRAAAEKLFGNME | |
| | GDCPSDWKTDSTCRMVTSESKNVKLTVS | |

Affinity variants of the generated human or mouse TfR antibodies were made by systematically introducing mutations into individual CDR of each antibody and the resulting variants were subjected to multiple rounds of selection with decreasing concentrations of antigen and/or increasing periods of dissociation to isolate clones with improved affinities. The sequences of individual variants were used to construct a combinatorial library which was subjected to an additional round of selection with increased stringency to identify additive or synergistic mutational pairings between the individual CDR regions. Individual combinatorial clones are sequenced. The heavy chain and light chain CDRs and VH/VL sequences of the human TfR binding domains are provided in Table 1a and Table 1b.

Human or mouse TfR binding antibody or antibody fragments were generated by recombinant DNA technology. Such TfR binding antibody or antibody fragments can be expressed in a mammalian cell line such as HEK293 or CHO, either transiently or stably transfected with an expression system using an optimal predetermined HC:LC vector ratio or a single vector system encoding both HC and LC. Clarified media, into which the protein has been secreted, can be purified using the commonly used techniques.

### Binding Affinity

Binding affinity and binding stoichiometry of the exemplified human TfR antibody or antibody fragments to human and cynomolgus TfR was characterized using a surface plasmon resonance assay on a Biacore 8K instrument primed with HBS-EP+ (10 mM Hepes pH7.4+150 mM NaCl+3 mM EDTA+0.05% (w/v) surfactant P20) running buffer and analysis temperature set at 37° C. Target human and cynomologus TfR ECD's were immobilized on a CM4 chip (Cytiva P/N 29104989) using standard NHS-EDC amine coupling. The TfR binding antibody or antibody fragments were prepared at a final concentration of 0.3, 0.1, 0.033, 0.01, 0.0033, 0.001, 0.00033, 0.0001 μM respectively by dilution of stock solution into running buffer.

Binding analysis was performed in a multi-cycle kinetics manner. Each analysis cycle consists of (1) injection of the lowest to highest concentration proteins over all Fc at 50 μL/min for 140 seconds followed by return to buffer flow for 400 seconds to monitor dissociation phase; (2) regeneration of chip surfaces with injection of 3M magnesium chloride, for 30 seconds at 100 μL/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 6a.

Alternatively, binding affinity of the exemplified human TfR antibody or antibody fragments to human TfR was characterized using a bio-layer interferometry assay (BLI) on an Octet BLI R8 instrument primed with HBS-EP+ (10 mM HEPES pH7.4+150 mM NaCl+3 mM EDTA+0.05% (w/v) tween-20) running buffer and analysis temperature set at 25° C. Human TfR antibody or antibody fragments were loaded onto an Octet AR2G biosensor (Sartorius P/N 18-5092) using standard sulfo-NHS/EDC amine coupling. Target human TfR ECD were prepared at a final concentration of 100, 25, 6.25 and 1.56 nM respectively by dilution of stock solution into running buffer in a 96-well black microplate (Greiner P/N 655209).

Binding analysis was performed in a multi-cycle kinetics manner. Each analysis cycle consists of (1) activation of the AR2G biosensor tips by tipping into premixed EDC/NHS solution for 300 seconds; (2) loading human TfR antibody or antibody fragments onto the activated biosensor tips by dipping into solution for 600 seconds; (3) quenching of the biosensor tip into 1M ethanolamine solution; (4) getting to a stable baseline by dipping into buffer wells for 60 seconds; (5) association step by dipping into wells containing serially diluted human TfR ECD for 120 seconds; (6) performing dissociation by dipping into buffer wells for 240 seconds; (7) lastly, regeneration of the tips were done for next cycle. Data were processed using standard double-referencing and fit to 1:1 binding model using Octet analysis studio software, to determine the association rate (Kₐ, M⁻¹s⁻¹ units), dissociation rate (K_{diss}, s⁻¹ units). The equilibrium dissociation constant (K_{D}) is calculated from the relationship K_{D}=k_{off}/kₒₙ, and is in molar units. Results are provided in Table 6b below.

TABLE 6a
Binding Affinity of Exemplified human TfR antibody or
antibody fragments to human or cynomolgus TfR at 37° C.

| Human | | | | |
|---|---|---|---|---|
| TfR | | Standard error | | Standard error |
| binding | | of the mean, | | of the mean, |
| antibody or | Human TfR K_{D} | Human TfR K_{D} | Cyno TfR K_{D} | Cyno TfR K_{D} |
| antibody | (Biacore, nM) | (Biacore, nM) | (Biacore, nM) | (Biacore, nM) |
| fragments | at 37° C. | n = 3 | at 37° C. | n = 3 |
| A4 | 0.522 | 0.284 | 502.210 | 8.129 |

TABLE 6b
Binding Affinity of Exemplified human TfR antibody
or antibody fragments to human TfR at 25° C.

| | | Standard | | | | |
|---|---|---|---|---|---|---|
| Human TfR | | error of the | | | | |
| binding | | mean, Human | | | | |
| antibody or | Human TfR | TfR K_{D} | | | | |
| antibody | K_{D} (BLI, nM) | (BLI, nM) | | | Kdiss | Kdiss |
| fragments | at 25° C. | n = 3 | Ka (1/Ms) | Ka Error | (1/s) | Error |
| A2 | 1.21 | 0.013 | 6.34E+05 | 3.21E+03 | 7.69E−04 | 7.50E−06 |
| A3 | 1.88 | 0.019 | 5.81E+05 | 3.59E+03 | 1.09E−03 | 9.28E−06 |
| A4 | 0.73 | 0.010 | 5.91E+05 | 2.38E+03 | 4.31E−04 | 5.79E−06 |
| A5 | 1.40 | 0.014 | 5.23E+05 | 2.31E+03 | 7.34E−04 | 6.30E−06 |

### Example 2: Synthesis and Characterization of dsRNAs Targeting SNCA

Single strands (sense and antisense) of the dsRNA duplexes were synthesized on solid support via a MerMade™ 12 (LGC Biosearch Technologies). The sequences of the sense and antisense strands were shown in Table 3a or 3b. Sense strands conjugated via the 3′-terminus were synthesized using phthalamido amino C6 lcaa CPG 500 Å (Chemgenes). Sense strands conjugated via the 5′-terminus were synthesized using a standard support (LGC Biosearch Technologies) and the Preparation 11 amidite for the final coupling. The antisense strands were synthesized using standard support (LGC Biosearch Technologies). The oligonucleotides were synthesized via phosphoramidite chemistry at either 5, 10, or 50 μmol scales.

Standard reagents were used in the oligo synthesis (Table 7), where 0.1M xanthane hydride in pyridine was used as the sulfurization reagent and 20% DEA in ACN was used as an auxiliary wash post synthesis. All monomers (Table 8) were made at 0.1M in ACN and contained a molecular sieves trap bag.

The oligonucleotides were cleaved and deprotected (C/D) at 45° C. for 20 hours. The sense strands were C/D from the CPG using cold 50% (methylamine/ammonia hydroxide 28-30%) at RT for 3 hrs, whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 μm PVDF syringeless filter, 0.22 μm PVDF Steriflip® vacuum filtration or 0.22 μm PVDF Stericup® Quick release. The CPG was back washed/rinsed with either 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using anion-exchange (AEX). For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. Fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LC/MS for mass purity and UPLC for UV-purity.

TABLE 7
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 8
Phosphoramidites

| Phosphoramidite | Abbreviation | Supplier | Catalog # | CAS |
|---|---|---|---|---|
| DMT-2′-F-A(Bz)-CE | fA | Hongene | PD1-001 | 136834-22-5 |
| Phosphoamidite | | | | |
| DMT-2′-F—C(Ac)—CE | fC | Hongene | PD3-001 | 159414-99-0 |
| Phosphoamidite | | | | |
| DMT-2′-F-G(iBu)-CE | fG | Hongene | PD2-002 | 144089-97-4 |
| Phosphoamidite | | | | |
| DMT-2′-F—U—CE | fU | Hongene | PD5-001 | 146954-75-8 |
| Phosphoamidite | | | | |
| DMT-2′-O—Me-A(Bz)-CE | mA | Hongene | PR1-001 | 110782-31-5 |
| Phosphoamidite | | | | |
| DMT-2′-O—Me—C(Ac)—CE | mC | Hongene | PR3-001 | 199593-09-4 |
| Phosphoamidite | | | | |
| DMT-2′-O—Me-G(iBu)-CE | mG | Hongene | PR2-002 | 150780-67-9 |
| Phosphoamidite | | | | |
| DMT-2′-O—Me—U—CE | mU | Hongene | PR5-001 | 110764-79-9 |
| Phosphoamidite | | | | |
| 5′bis(POM) vinyl | POM-VPmU | Hongene | PR5-032 | BVPMUP23B2A1 |
| phosphate-2′-Ome-U3′CE | | | | |
| phosphoroamidite | | | | |
| Reverse Abasic | iAb | Chemgenes | ANP-1422 | 401813-16-9 |
| phosphoroamidite | | | | |
| Abasic phosphoroamidite | Aba | Chemgenes | ANP-7058 | 129821-76-7 |
| 2-Cyanoethyl | | Lilly | | |
| ((2R,3R,4R,5R)-5-(2,4- | | | | |
| dioxo-3,4- | | | | |
| dihydropyrimidin-1(2H)- | | | | |
| yl)-2-((1,3-dioxoisoindolin- | | | | |
| 2-yl)methyl)-4- | | | | |
| methoxytetrahydrofuran-3- | | | | |
| yl) | | | | |
| diisopropylphosphoramidite | | | | |

### Example 3: Generation of Conjugates Comprising TfR Binding Antibody or Antibody Fragments

Certain abbreviations are defined as follows: “ACN” refers to acetonitrile; “AS” refers to antisense strand; “C/D” refers to cleaved and deprotected; “DAR” refers to drug/siRNA to antibody/protein ratio; “DCM” refers to dichloromethane; “DEA” refers to diethylamine; “DIAD” refers to diisopropyl azodicarboxylate; “DIEA” refers to N,N-diisopropylethylamine; “DMT” refers to dimethoxytrityl; “dsRNA” refers to double stranded ribonucleic acid; “EDCI” refers to 1-ethyl-3-(3-dimethylaminopropyl) carbodiimide; “EtOAc” refers to ethyl acetate; “EtOH” refers to ethanol and ethyl alcohol; “h” refers to hours; “IP-RP” refers to ion-pair reverse phase; “LC/MS” refers to liquid chromatography mass spectrometry; “LTQ/MS” refers to linear ion trap mass spectrometer; “MeOH” refers to methanol and methyl alcohol; “min” refers to minutes; “MW” refers to molecular weight; “NHS” refers to N-hydroxysuccinimide; “OD” refers to optical density; “PBS” phosphate-buffered saline; “rpm” refers to revolutions per minute; “siRNA” refers to small interfering RNA; “SMCC” refers to succinimidyl-4-(N-maleimidomethyl)cyclohexane-1-carboxylate; “SS” refers to sense strand; “TEA” refers to triethylamine; “TFA” refers to trifluoroacetic acid; “THF” refers to tetrahydrofuran; “UPLC” refers to ultra-performance liquid chromatography; and “UV” refers to ultraviolet.

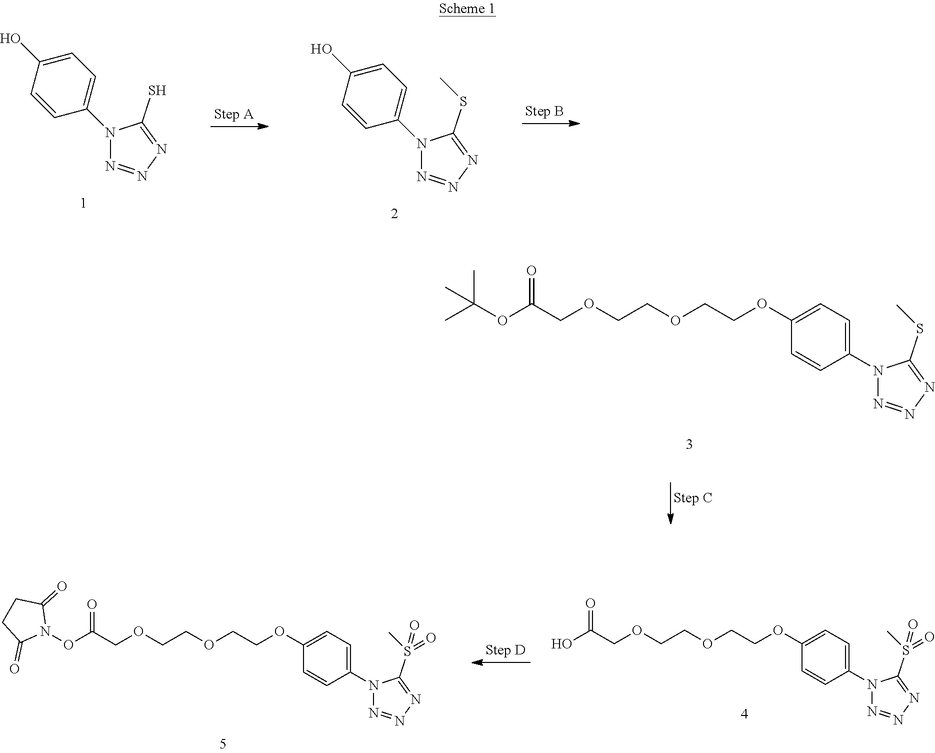 [SMILES: Oc1ccc(-n2nnnc2S)cc1 ; CSc1nnnn1-c1ccc(O)cc1 ; CSc1nnnn1-c1ccc(OCCOCCOCC(=O)OC(C)(C)C)cc1 ; CS(=O)(=O)c1nnnn1-c1ccc(OCCOCCOCC(=O)ON2C(=O)CCC2=O)cc1 ; CS(=O)(=O)c1nnnn1-c1ccc(OCCOCCOCC(=O)O)cc1]

Scheme 1, step A depicts the methylation of the thiol on compound (1) using iodomethane and a suitable base such as DIEA in a solvent such as THF to give compound (2). Step B shows an alkylation of compound (2) with tert-butyl 2-(2-(2-bromoethoxy) ethoxy)acetate using a base such as potassium carbonate in a solvent such as acetone to give compound (3). Step C shows the oxidation of compound (3) with hydrogen peroxide and ammonium molybdate (VI) tetrahydrate in a solvent such as EtOH followed by an acidic deprotection using an acid such as TFA in a solvent such as DCM to give compound (4). Note that in the case of the 1H-tetrazole, the deprotection took place during the oxidation step. Step D depicts a coupling of compound (4) and 1-hydroxypyrrolidine-2,5-dione using EDCI in a solvent system such as DCM and THF to give compound (5).

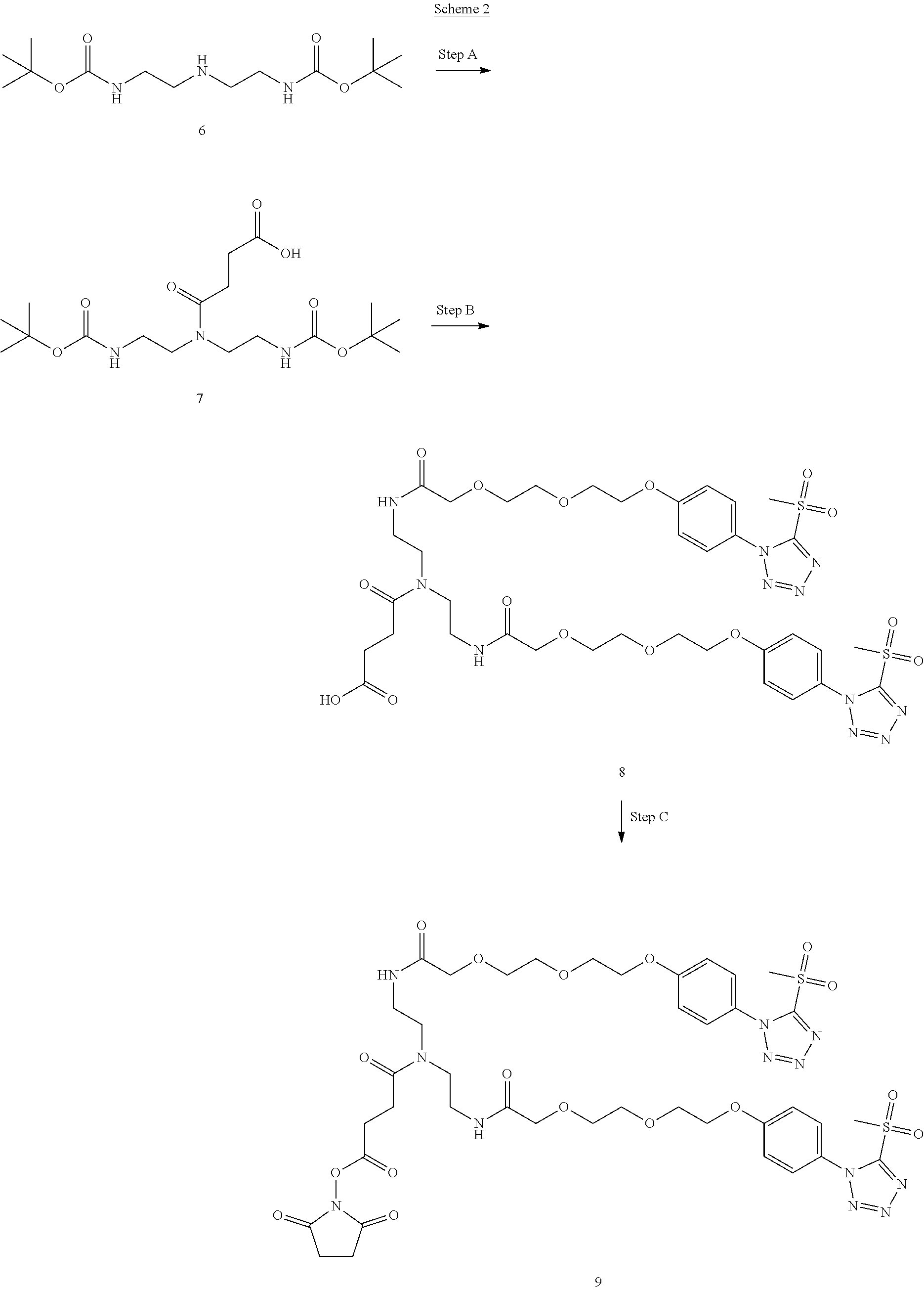 [SMILES: CC(C)(C)OC(=O)NCCNCCNC(=O)OC(C)(C)C ; CC(C)(C)OC(=O)NCCN(CCNC(=O)OC(C)(C)C)C(=O)CCC(=O)O ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)O)cc1 ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

Scheme 2, step A shows the addition of succinic anhydride to compound (6) in a solvent such as DCM to give compound (7). Step B depicts the acidic deprotection of compound (7) using an acid such as TFA in a solvent such as DCM followed by a coupling with compound (5) using a suitable base such as DIEA in a solvent such as DMF to give compound (8). Step C depicts a coupling of compound (8) and 1-hydroxypyrrolidine-2,5-dione using EDCI in a solvent system such as DCM to give compound (9).

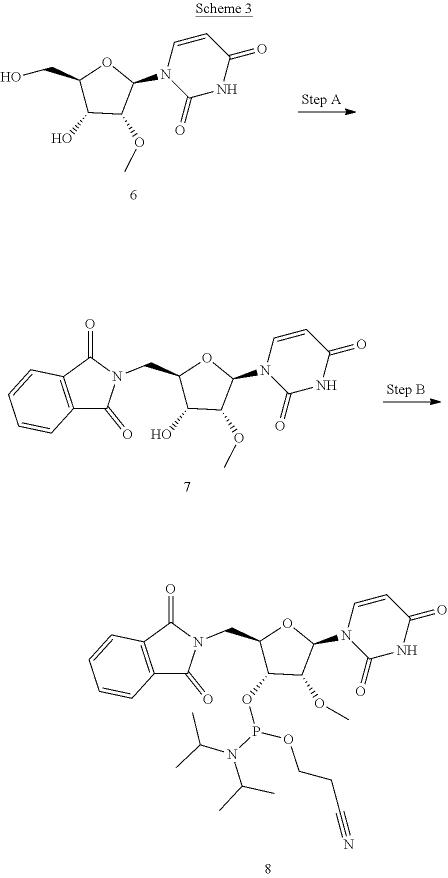 [SMILES: CO[C@@H]1[C@H](O)[C@@H](CO)O[C@H]1n1ccc(=O)[nH]c1=O ; CO[C@@H]1[C@H](O)[C@@H](CN2C(=O)c3ccccc3C2=O)O[C@H]1n1ccc(=O)[nH]c1=O ; CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

Scheme 3, step A shows the coupling of compound (10) and isoindoline-1,3-dione using DIAD and tributyl phosphine in a solvent such as THF to give compound (11). Step B depicts the phosphorylation of compound (11) with 2-cyanoethyl-N,N-diisopropylchlorophosphoramidite using a base such as DIEA in a solvent such as DCM to give compound (12).

### Preparation 1

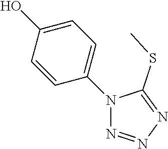

### 4-(5-(Methylthio)-1H-tetrazol-1-yl) phenol

A solution of 4-(5-mercapto-1H-tetrazol-1-yl) phenol (4.00 g, 20.6 mmol) in THF (50 mL) was cooled to 0° C. DIEA (4.31 g, 33.3 mmol) was added then stirred for 10 minutes before adding iodomethane (1.54 mL, 24.7 mmol) dropwise over a period of 1 minute. The mixture was stirred at 0° C. for 20 minutes, and then stirred at ambient temperature for 12 hours. After this time, the mixture was diluted with EtOAc (100 mL) and washed with saturated aqueous NH₄Cl (2× 50 mL). The organic layer was separated, dried over sodium sulfate, and concentrated in vacuo to give the title compound (4.2 g, 93%). ES/MS m z: 209 (M+H).

### Preparation 2

### tert-Butyl 2-(2-(2-(4-(5-(methylthio)-1H-tetrazol-1-yl) phenoxy) ethoxy) ethoxy)acetate

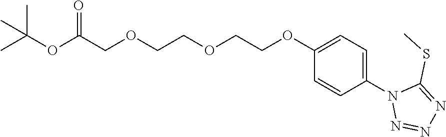 [SMILES: CSc1nnnn1-c1ccc(OCCOCCOCC(=O)OC(C)(C)C)cc1]

In a pressure vessel, potassium carbonate (3.15 g, 22.8 mmol) was added to tert-butyl 2-(2-(2-bromoethoxy) ethoxy)acetate (4.33 g, 14.8 mmol) and 4-(5-(methylthio)-1H-tetrazol-1-yl) phenol (2.5 g, 11.4 mmol) in acetone (60 mL). The pressure vessel was sealed and heated at 80° C. for 8 hours with vigorous stirring. After this time, the mixture was cooled to ambient temperature then filtered while washing through with acetone/EtOAc/DCM (30 mL each). The filtrate was concentrated in vacuo and purified via silica gel column chromatography eluting with 0-100% EtOAc/DCM to give the title compound as a white solid (3.98 g, 85%). ES/MS m z: 411 (M+H).

### Preparation 3

### 2-(2-(2-(4-(5-(Methylsulfonyl)-1H-tetrazol-1-yl) phenoxy) ethoxy) ethoxy) acetic acid

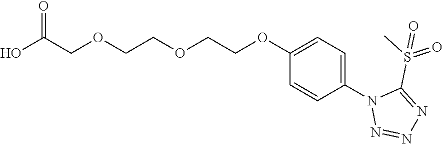 [SMILES: CS(=O)(=O)c1nnnn1-c1ccc(OCCOCCOCC(=O)O)cc1]

tert-Butyl 2-(2-(2-(4-(5-(methylthio)-1H-tetrazol-1-yl) phenoxy) ethoxy) ethoxy)acetate (3.98 g, 9.21 mmol) was dissolved in EtOH (100 mL) and cooled to 5-10° C. Then, 30% hydrogen peroxide (19 mL, 184 mmol) was added, followed by ammonium molybdate (VI) tetrahydrate (1.14 g, 0.921 mmol). The mixture was allowed to warm to ambient temperature and then stirred for 4 hours, after which it was diluted with DCM (150 mL) and washed with saturated aqueous sodium chloride solution. The organic phase was separated, dried over sodium sulfate, and concentrated in vacuo. The resulting residue was purified via silica gel column chromatography eluting with 0-100% EtOAc/DCM to give the title compound as a white solid (3.00 g, 80%). ES/MS m z: 385 (M−H).

### Preparation 4

### 2,5-Dioxopyrrolidin-1-yl 2-(2-(2-(4-(5-(methylsulfonyl)-1H-tetrazol-1-yl) phenoxy) ethoxy) ethoxy)acetate

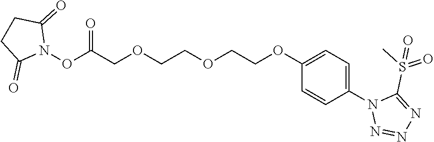 [SMILES: CS(=O)(=O)c1nnnn1-c1ccc(OCCOCCOCC(=O)ON2C(=O)CCC2=O)cc1]

EDCI (1.60 g, 10.3 mmol) was added to a solution of 2-(2-(2-(4-(5-(methylsulfonyl)-1H-tetrazol-1-yl) phenoxy) ethoxy) ethoxy) acetic acid (2.80 g, 7.25 mmol) and 1-hydroxypyrrolidine-2,5-dione (1.33 g, 11.6 mmol) in DCM (50 mL) and THF (70 mL). Another 20 mL of DCM was added to bring the mixture into a solution followed by stirring at ambient temperature for 12 hours. After this time, concentrated in vacuo and purified via silica gel column chromatography eluting with 0-100% EtOAc/DCM to give the title compound (2.61 g, 65%). ES/MS m z: 484 (M+H).

### Preparation 5

### 4-(Bis(2-((tert-butoxycarbonyl)amino) ethyl)amino)-4-oxobutanoic acid

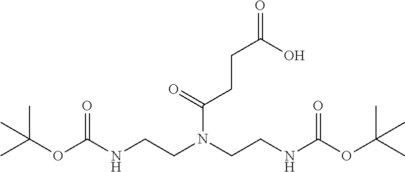 [SMILES: CC(C)(C)OC(=O)NCCN(CCNC(=O)OC(C)(C)C)C(=O)CCC(=O)O]

Under a N₂ atmosphere, a solution of succinic anhydride (989.5 mg, 9.88 mmol) in DCM (40 mL) was added over 30 minutes via addition funnel to a solution of di-tert-butyl (azanediylbis (ethane-2,1-diyl))dicarbamate (3000 mg, 9.88 mmol) in DCM (40 mL) at ambient temperature. The mixture was stirred for 48 hours then diluted with water (50 mL) and extracted with DCM (3×40 mL). The organic layers were combined, washed with saturated aqueous NH₄Cl solution, aqueous sodium bicarbonate, then saturated aqueous NaCl. The organics were then dried over MgSO₄, filtered, and concentrated in vacuo. The resulting residue was diluted with DCM (50 mL), absorbed onto silica gel, and concentrated in vacuo. The residue was purified by silica gel chromatography eluting with 10% MeOH/DCM to give the title compound as a white solid (2.84 g, 70%). ES/MS m z: 402 (M−H).

### Preparation 6

### 1-(4-(5-(Methylsulfonyl)-1H-tetrazol-1-yl) phenoxy)-12-(2-(2-(2-(2-(4-(5-(methylsulfonyl)-1H-tetrazol-1-yl) phenoxy) ethoxy) ethoxy) acetamido)ethyl)-8,13-dioxo-3,6-dioxa-9,12-diazahexadecan-16-oic acid

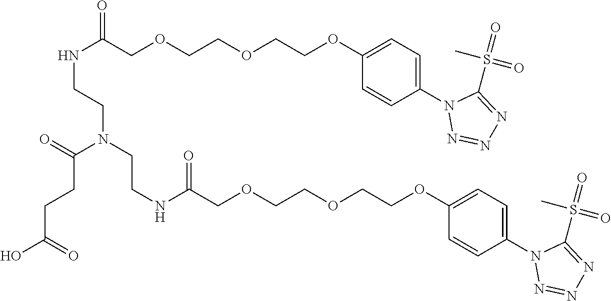 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)O)cc1]

TFA (2 mL) was added to a solution of 4-(bis(2-((tert-butoxycarbonyl)amino) ethyl)amino)-4-oxobutanoic acid (470 mg, 1.05 mmol) in DCM (3 mL) and then stirred at ambient temperature for 3 hours. The mixture was then concentrated in vacuo, further azeotroped with toluene (2×10 mL), and placed under high vacuum for 2 hours to give 4-(bis(2-(12-azaneyl)ethyl)amino)-4-oxobutanoic acid as a TFA salt. This material was dissolved in DMF (5 mL) and DIEA (407 mg, 3.15 mmol) was added followed by a solution of 2,5-dioxopyrrolidin-1-yl 2-(2-(2-(4-(5-(methylsulfonyl)-1H-tetrazol-1-yl) phenoxy) ethoxy) ethoxy)acetate (1.07 g, 2.10 mmol) in DMF (4 mL). The mixture was stirred at ambient temperature for 1 hour, then diluted with DCM (100 mL), and washed with saturated aqueous NaCl and water. The organics were dried over sodium sulfate, filtered, and concentrated in vacuo. The residue was purified by silica gel chromatography eluting with 0-20% MeOH/DCM to give the title compound as a white solid (855 mg, 82%). ES/MS m z: 940 (M+H).

### Preparation 7

### 2,5-Dioxopyrrolidin-1-yl 1-(4-(5-(methylsulfonyl)-1H-tetrazol-1-yl) phenoxy)-12-(2-(2-(2-(2-(4-(5-(methylsulfonyl)-1H-tetrazol-1-yl) phenoxy) ethoxy) ethoxy) acetamido)ethyl)-8,13-dioxo-3,6-dioxa-9, 12-diazahexadecan-16-oate

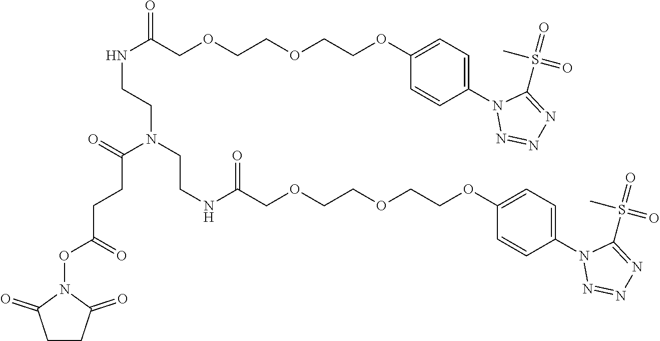 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

EDCI (72.7 mg, 468 μmol) was added to a solution of 1-(4-(5-(methylsulfonyl)-1H-tetrazol-1-yl) phenoxy)-12-(2-(2-(2-(2-(4-(5-(methylsulfonyl)-1H-tetrazol-1-yl) phenoxy) ethoxy) ethoxy) acetamido)ethyl)-8, 13-dioxo-3,6-dioxa-9,12-diazahexadecan-16-oic acid (220 mg, 234 μmol) and 1-hydroxypyrrolidine-2,5-dione (53.9 mg, 468 μmol) in DCM (3 mL) then stirred at ambient temperature for 12 hours. The mixture was concentrated in vacuo and purified by silica gel chromatography eluting with 0-50% MeOH/DCM to give the title compound as a white solid (206 mg, 79%). ES/MS m z: 1037 (M+H).

### Preparation 8

### 2-(((2R,3R,4R,5R)-5-(2,4-Dioxo-3,4-dihydropyrimidin-1 (2H)-yl)-3-hydroxy-4-methoxytetrahydrofuran-2-yl)methyl) isoindoline-1,3-dione

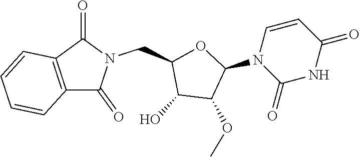 [SMILES: CO[C@@H]1[C@H](O)[C@@H](CN2C(=O)c3ccccc3C2=O)O[C@H]1n1ccc(=O)[nH]c1=O]

A solution of 1-((2R,3R,4R,5R)-4-hydroxy-5-(hydroxymethyl)-3-methoxytetrahydrofuran-2-yl)pyrimidine-2,4 (1H,3H)-dione (30 g, 120 mmol), isoindoline-1,3-dione (21 g, 140 mmol), DIAD (27 mL, 140 mmol), tributyl phosphine (36 mL, 150 mmol), and THF (300 mL) was stirred at ambient temperature for 12 h. The crude reaction was filtered, concentrated in vacuo, and purified via silica gel flash chromatography eluting with 0-100% EtO Ac/hexanes to give the title compound as a white solid (6.0 g, 13%).

### Preparation 9

### 2-Cyanoethyl((2R,3R,4R,5R)-5-(2,4-dioxo-3,4-dihydropyrimidin-1 (2H)-yl)-2-((1,3-dioxoisoindolin-2-yl)methyl)-4-methoxytetrahydrofuran-3-yl)diisopropylphosphoramidite

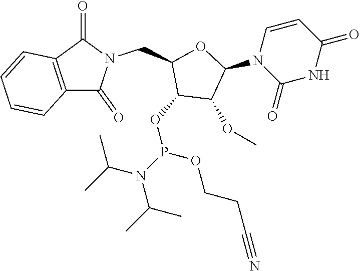 [SMILES: CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

A solution of 2-(((2R,3R,4R,5R)-5-(2,4-dioxo-3,4-dihydropyrimidin-1 (2H)-yl)-3-hydroxy-4-methoxytetrahydrofuran-2-yl)methyl) isoindoline-1,3-dione (3.00 g, 7.74 mmol), 2-cyanoethyl-N,N-diisopropylchlorophosphoramidite (2.47 mL, 11.6 mmol), DIEA (4.05 mL, 23.2 mmol), and DCM (40 mL) was stirred at ambient temperature. After 1 hour, additional 2-cyanoethyl-N,N-diisopropylchlorophosphoramidite (0.82 mL, 3.8 mmol) was added. After 1 hour, the crude reaction was poured into a slurry of silica gel (15 g) in 30 mL of 1% TEA/DCM, concentrated in vacuo to a dry powder, and purified via silica gel flash chromatography eluting with 40-100% EtOAc/hexanes (0.5% TEA) to give the title compound as a white foam (3.70 g, 81%). 1H NMR (d6-DMSO) d 11.4 (br s, 1H), 7.96-7.78 (m, 5H), 5.83 (dd, 1H), 5.71 (dd, 1H), 4.46-3.47 (m, 9H), 3.39 (s, 1.5H), 3.35 (s, 1.5H), 2.82-2.73 (m, 2H), 1.16-0.97 (m, 12H). 31p NMR (d6-DMSO) d 149.7, 149.4.

### Preparation 10

### 3′ Bis-tetrazole linker-functionalized sense strand

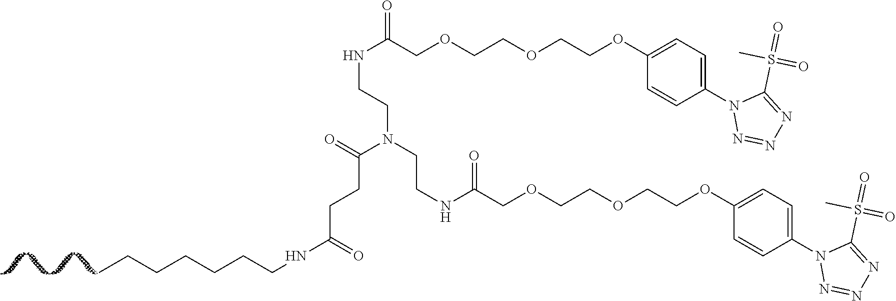 [SMILES: CCCCCCCNC(=O)CCC(=O)N(CCNC(=O)COCCOCCOc1ccc(-n2nnnc2S(C)(=O)=O)cc1)CCNC(=O)COCCOCCOc1ccc(-n2nnnc2S(C)(=O)=O)cc1]

A Falcon tube was charged with SNCA-DV22-SS-3C₆ (2.00 mL, 1694 μM) and 2 mL of PBS 7.4. 2,5-Dioxopyrrolidin-1-yl 1-(4-(5-(methylsulfonyl)-1H-tetrazol-1-yl) phenoxy)-12-(2-(2-(2-(2-(4-(5-(methylsulfonyl)-1H-tetrazol-1-yl) phenoxy) ethoxy) ethoxy) acetamido)ethyl)-8, 13-dioxo-3,6-dioxa-9,12-diazahexadecan-16-oate (850 μL, 20 mM in ACN) was added. The mixture was vortexed for 3 minutes, then shook at 900 rpm at 40° C. for 1.5 hours. After this time, added more 2,5-dioxopyrrolidin-1-yl 1-(4-(5-(methylsulfonyl)-1H-tetrazol-1-yl) phenoxy)-12-(2-(2-(2-(2-(4-(5-(methylsulfonyl)-1H-tetrazol-1-yl) phenoxy) ethoxy) ethoxy) acetamido)ethyl)-8,13-dioxo-3,6-dioxa-9,12-diazahexadecan-16-oate (250 μL, 20 mM in ACN) and ACN (400 μL), continued to shake for 2 additional hours at 40° C. then stored at 4° C. The excess ACN was removed on Genevac, then added 5 mL of water, followed by de-salting using a 3K spin filter (Fisher biologics, 4500 rpm, 10 minutes). Nano drop measurement: concentration=12170 ng/μL, 1510 μmol/L, total 1.65 mL.

The compound in Table 9 below was prepared in a manner essentially analogous to that found in Preparation 10.

TABLE 9

| Prep | Name | Structure |
|---|---|---|
| 11 | 3′ Tetrazole- linked functionalized sense strand | 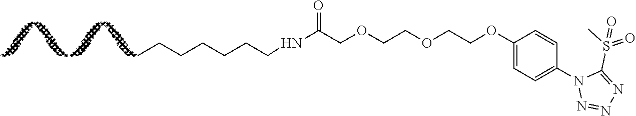 [SMILES: CCCCCCCNC(=O)COCCOCCOc1ccc(-n2nnnc2S(C)(=O)=O)cc1] |

*12.85 OD/mL, concentration = 626 umol/L, 4.71 mg/mL, total 1.65 mL, 7.80 mg

### Preparation 12

### 5′ Tetrazole-linked functionalized sense strand

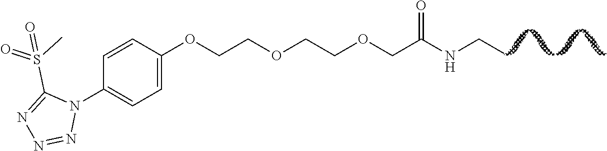 [SMILES: CCNC(=O)COCCOCCOc1ccc(-n2nnnc2S(C)(=O)=O)cc1]

A sense strand (0.0011 mmol in 0.395 mL water) synthesized using conditions found in the protocols below was added to 20× borate buffer (0.059 mL), then was treated with a solution of 2,5-dioxopyrrolidin-1-yl 2-(2-(2-(4-(5-(methylsulfonyl)-1H-tetrazol-1-yl) phenoxy) ethoxy) ethoxy)acetate (0.0053 g, 0.0110 mmol) in MeCN (0.395 mL). The solution was shaken for 30 mins at 40° C. The solution was then diluted to 20 mL using RNAse free water to bring concentration of organic solvent to <10%. Excess NHS ester was removed using 20 mL 3K MWCO centrifugal spin tubes at 3500×g for ˜30 minutes. The oligonucleotides were rinsed with RNAse free water three times. After removing NHS ester, 1 mL of RNAse free water was added then aspirated 10× and the retentate was transferred to a 5 mL falcon tube. This was repeated until complete transfer of oligo by measuring concentration of compound on filter via nanodrop. The final oligonucleotide was analyzed for concentration (nano drop at A260), characterized by IP-RP, LCMS for mass purity, and UPLC for UV-purity. ES/MS (m/z): 7324.04 (M+H).

### Linker-SNCA Duplex

The nanodrop concentrations of aqueous solutions of each strand (average of 3×) were measure as SS=1322 μM and AS=1108 μM. The sense strand and antisense strand were annealed to form a dsRNA. 32 mL of SS and 36.2 mL of AS were mixed and shook for 30 min at 30° C. The amount of residual SS strand was measured until completion and required adding an additional 360 μL of AS. Removed endotoxins by filtering through a 0.45 μM filter. The resulting 75 mL of solution measured (Nanodrop™ Lite, 5× average, 10× dilution) 217 OD/mL equating to 575 μM and a total of 653 mg. LTQ/MS m z 7358,7825; UV purity 99+%.

### Bis-Linker-SNCA Duplex

The nanodrop concentrations of aqueous solutions of each strand (average of 3×) were measured as SS=1510 μM and AS=2074 μM. The sense strand and antisense strand were annealed to form a dsRNA. 1.2 mL of SS and 0.84 mL of AS were mixed and shook for 10 min at 30° C. The amount of residual SS strand was measured by UPLC until completion. The resulting 2.04 mL of solution measured (Nanodrop™ Lite, 5× average, 10× dilution) 217 OD/mL equating to 834 μM. LTQ/MS m z 8061,7826; UV purity 87+%.

Conjugation of dsRNA to TfR Binding Antibody or Antibody Fragments

Site-specific native or engineered cysteine amino acid residues in the TfR binding antibody or antibody fragments were used to conjugate dsRNA. Cysteines can be engineered into the primary amino acid sequence of the TfR binding antibody or antibody fragments. The approach of introducing cysteines as a means for conjugation has been described in WO 2018/232088, which is both incorporated by reference in its entirety and incorporated specifically in relation to conjugation via cysteine residues. For engineered cysteine conjugation, the TfR binding antibody or antibody fragments were first reduced with 40 molar equivalents reducing agent dithiothreitol (DTT) at 37° C. for two hours, followed by desalting to remove reducing agent via dialysis or desalting columns. This was followed by re-oxidation of the TfR binding antibody or antibody fragment to reform the structural disulfides with 10 molar equivalent dehydroascorbic acid (DHAA) incubation at ambient temperature for two hours. A follow up desalting was performed to remove oxidizing agent. For conjugation at the hinge disulfide or the c-termini of the heavy and light chain, where these site is the only conjugatable cysteine present in the molecule, TfR binding antibody or antibody fragments were reduced with 10 molar equivalents reducing agent tris(2-carboxyethyl) phosphine (TCEP). Following which, Bis-MSPT functionalized siRNA were conjugated onto the antibody or antibody fragment.

Conjugation of dsRNA onto TfR binding antibody or antibody fragments were done using the following methods.

### Conjugation Scheme 1

The conjugation method utilized the 3′SS tetrazole (MSPT)-functionalized dsRNA for conjugating onto the engineered cysteine of the TfR binding antibody or antibody fragments. For this method, TfR binding antibody or antibody fragment was prepared similarly as above to make the engineered thiol available for conjugation by undergoing a reduction and oxidation process of the TfR binding antibody or antibody fragments. This was followed by incubating the MSPT-dsRNA with the TfR binding antibody or antibody fragments at 1.2 to 2 molar equivalents for overnight conjugation at ambient temperature.

Conjugation was monitored using analytical anion exchange chromatography. A ProPac™ SAX-10 HPLC Column, 10 μm particle, 4 mm diameter, 250 mm length was utilized with the following method. Flow rate of 1 mL/min, Buffer A: 20 mM TRIS pH 7.0, Buffer B: 20 mM TRIS pH 7.0+1M NaCl, at ambient temperature.

TABLE 10a
HPLC gradient used to assess dsRNA conjugation
to TfR binding antibody or antibody fragment

| Time [min] | A [%] | B [%] |
|---|---|---|
| 0.00 | 85.0 | 15.0 |
| 8.00 | 0.0 | 100.0 |
| 9.00 | 0.0 | 100.0 |
| 9.10 | 85.0 | 15.0 |
| 10.0 | 85.0 | 15.0 |

### Conjugation Scheme 2

The conjugation method utilized the 3′SS bis-tetrazole (bis-MSPT)-functionalized dsRNA for conjugating onto the engineered cysteine of the TfR binding antibody or antibody fragments. For this method, TfR binding antibody or antibody fragment was prepared similarly as above to make the thiols at the hinge or at the c-termini of the heavy chains and light chains available for conjugation by undergoing a reduction process of the TfR binding antibody or antibody fragments. This was followed by incubating the bis-MSPT-dsRNA with the TfR binding antibody or antibody fragments at 1.2 to 2 molar equivalents for overnight conjugation at ambient temperature.

Synthesis and conjugation of conjugates comprising dsRNA and TfR binding antibody or antibody fragments via SMCC linker were done as described in WO2024/036096.

Conjugation was monitored using analytical anion exchange chromatography. A ProPac™ SAX-10 HPLC Column, 10 μm particle, 4 mm diameter, 250 mm length was utilized with the following method. Flow rate of 1 mL/min, Buffer A: 20 mM TRIS pH 7.0, Buffer B: 20 mM TRIS pH 7.0+1M NaCl, at ambient temperature.

TABLE 10b
HPLC gradient used to assess dsRNA conjugation
to TfR binding antibody or antibody fragment

| Time [min] | A [%] | B [%] |
|---|---|---|
| 0.00 | 85.0 | 15.0 |
| 8.00 | 0.0 | 100.0 |
| 9.00 | 0.0 | 100.0 |
| 9.10 | 85.0 | 15.0 |
| 10.0 | 85.0 | 15.0 |

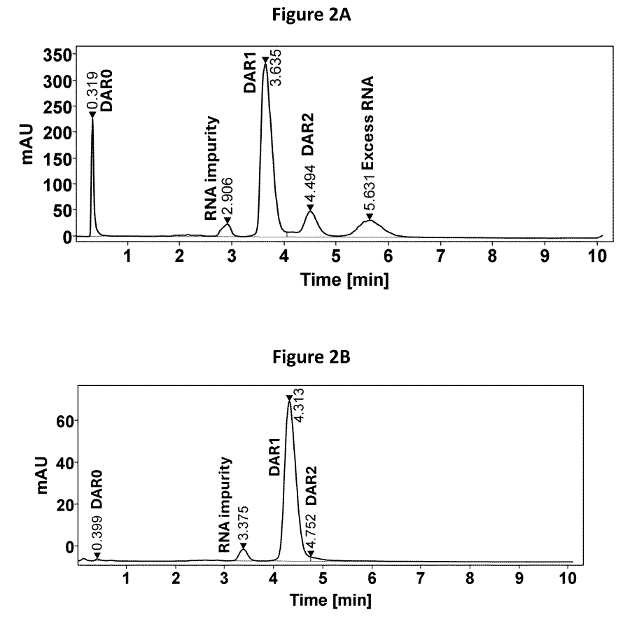

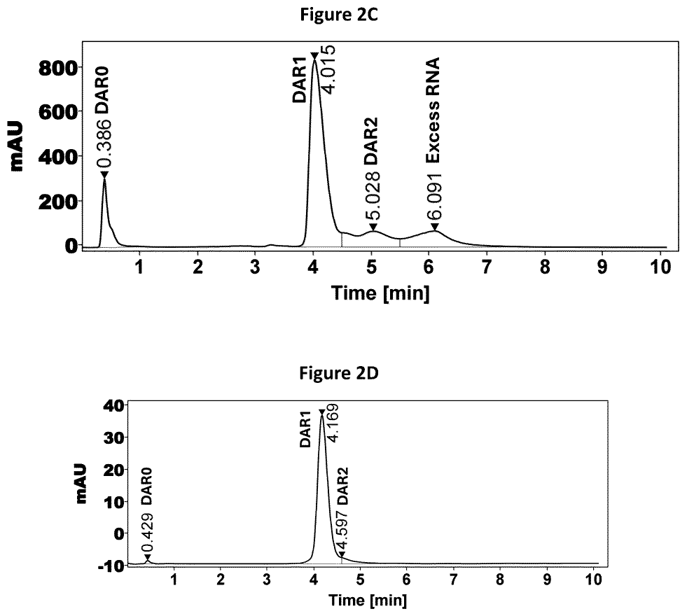

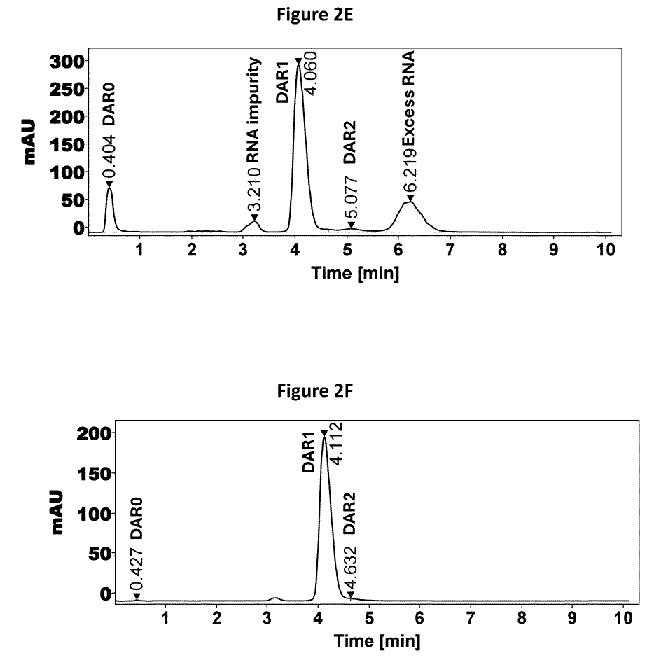

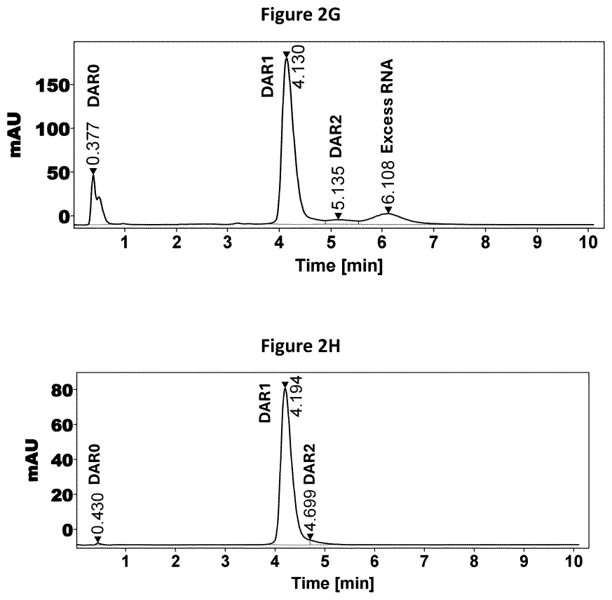

Drug/siRNA to antibody/protein ratio (DAR) was calculated based on peak area % from the analytical anion exchange (aAEX) chromatogram. See FIGS. 2A-2H.

Post conjugation of dsRNA to the TfR binding antibody or antibody fragment, excess dsRNA and unconjugated protein was removed by further purification. Either preparative size exclusion chromatography (SEC) or preparative anion exchange chromatography was utilized for purification of the final conjugate. Preparative SEC was performed using Cytiva Superdex® 200 in 1×PBS pH 7.2 under an isocratic condition. Alternatively, anion exchange, e.g., ThermoFisher POROS™ XQ, was used with starting buffer of 20 mM TRIS pH 7.0 and eluting with 20 column volume gradient with a buffer containing 20 mM TRIS pH 7.0 and 1M NaCl. These resulted in purified TfR binding antibody or antibody fragment-dsRNA conjugate devoid of excess dsRNA and minimal unconjugated protein. The resulting conjugate profile was analyzed by analytical anion exchange for final DAR quantitation (see Table 11).

TABLE 11
siRNA/drug to antibody ratio (DAR)

| | Average | % of | % of | % of |
|---|---|---|---|---|
| | DAR | DAR0 | DAR1 | DAR2 |
| A4-MSPT-dsRNA | 1.02 | 0.39 | 96.17 | 3.44 |
| No. 4 | | | | |
| A5-bis-MSPT- | 1.03 | 0.00 | 97.00 | 3.00 |
| dsRNA No. 4 | | | | |
| A2-MSPT-dsRNA | 1.01 | 0.00 | 99.00 | 1.00 |
| No. 4 | | | | |
| A3-bis-MAPT- | 1.01 | 1.00 | 97.10 | 1.9 |
| dsRNA No. 4 | | | | |

### Example 4: Ex Vivo Plasma Stability

The conjugates were subjected to ex vivo plasma stability assessment to evaluate stability of the conjugates and any dissociation of the siRNA from the antibody or antibody fragment. The conjugates were incubated in mouse or cynomolgus monkey plasma at 37° C. for 0, 24 or 48 hours respectively with rotation at 5 rpm. Antibody or antibody fragment was immunoprecipitated from the plasma sample using biotinylated goat anti-human IgG. The solution was then incubated with streptavidin beads at ambient temperature with rotation for 30 minutes. Following multiple washing steps with 1×PBS, the sample was eluted with 1% formic acid with 20% acetonitrile elution buffer by mixing at 2000 rpm for 15 seconds followed by 5-minute static benchtop hold. The eluted sample was then injected into LC-MS for analysis.

### LCMS Method:

Instrument: Sciex I.
Column: Agilent, PLRP-S 1000A 5 μM 50×1.0 MM, PN: PL1312-1502.
Injection Volume: 20 μL.
Column Temperature: 80° C.
Auto sampler Temperature: 5° C.
Mobile Phase A: water with 0.05% TFA.
Mobile Phase B: acetonitrile with 0.05% TFA.
Gradient:

| | | | | Max. Pressure |
|---|---|---|---|---|
| Time(min) | A(%) | B(%) | Flow(mL/min) | Limit(bar) |
| 0 | 98 | 2 | 0.4 | 1300 |
| 1.2 | 90 | 10 | 0.4 | 1300 |
| 2.1 | 72 | 28 | 0.4 | 1300 |
| 7.1 | 30 | 70 | 0.4 | 1300 |
| 7.2 | 10 | 90 | 0.4 | 1300 |
| 8 | 10 | 90 | 0.4 | 1300 |
| 8.1 | 98 | 2 | 0.4 | 1300 |
| 100 | 98 | 2 | 0.4 | 1300 |

MS: m/z 2000-5000

The results are shown in FIG. 4, which shows the conjugates comprising the 3′ mono-MSPT linker or 3′ bis-MSPT linker have increased plasma stability when compared to the conjugates comprising the SMCC linker.

### Example 5: In Vitro Characterization of the Human TfR Binding Proteins-dsRNA Conjugates

### In Vitro Potency Assessment in SYSY5Y Cells

SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with additives including 10% fetal bovine serum. Cells were incubated at 37° C. in a humidified atmosphere of 5% CO₂. On day one, SH-SY5Y cells were plated in fibronectin coated tissue culture plates and allowed to attach overnight. On day two, complete media was removed and replaced with RNAi agent in serum free media. Cells were incubated with RNAi agent for 7 days before analysis of gene mRNA expression. RT-qPCR was performed to quantify targeted mRNA levels using TaqMan™ Fast Advanced Cell-to-CT kit following the manufacturer's protocol (ThermoFisher A35377). The delta-delta CT method of normalizing to a housekeeping gene, ACTB (ThermoFisher, Hs99999903_g1, ACTB; Hs00240907_m1, SNCA), was used to determine relative amounts of gene mRNA expression. A three or four parameter logistic fit was used to determine IC50.

Results provided in Table 12 demonstrate exemplified human TFR binding protein-siRNA conjugates provide potency for knocking down human SNCA gene.

TABLE 12
In vitro potency for reducing human
SNCA mRNA in SH-SY5Y cells

| | IC50 (nM) |
|---|---|
| A4-MSPT-dsRNA No. 4 | 2.403 |
| A5-bis-MSPT-dsRNA No. 4 | 6.042 |
| A2-MSPT-dsRNA No. 4 | 11.21 |
| A3-bis-MAPT-dsRNA No. 4 | 2.912 |

### Example 6: In Vivo Characterization of the Human TfR Binding Antibody or Antibody Fragments-dsRNA Conjugates in Human TfR (hTfR) Transgenic Mice

The pharmacodynamic efficacy of the human TfR binding antibody or antibody fragment-SNCA dsRNA conjugates were evaluated in human TfR transgenic mice. The conjugates were dosed in hTfR transgenic mice by a single IV injection at 0.25 mg/kg of siRNA concentration and compared to PBS dosed group (n=4 each group). For takedowns, deeply anesthetized animals underwent cardiac perfusion 28 days following IV dosing, then brain tissues were collected and processed for RT-qPCR in tissue homogenates.

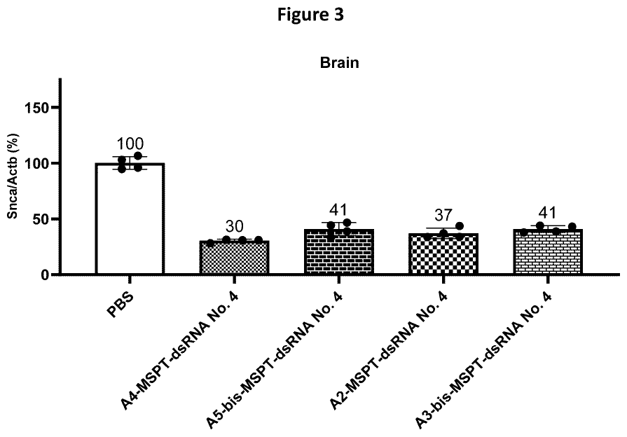

As shown in FIG. 3, all the TfR antibody or antibody fragment-SNCA dsRNA conjugate demonstrated significant reduction of SNCA mRNA in brain compared to the PBS treated group. Specifically, treatment with A4-MSPT-dsRNA No. 4 conjugate resulted in 30% SNCA mRNA remaining (70% knock down), A5-bis-MSPT-dsRNA No. 4 conjugate resulted in 41% SNCA mRNA remaining (59% knock down), A2-MSPT-dsRNA No. 4 conjugate resulted in 37% SNCA mRNA remaining (63% knock down), A3-bis-MSPT-dsRNA No. 4 conjugate resulted in 41% SNCA mRNA remaining (59% knock down).

SEQUENCE LISTING

| SEQ | |
|---|---|
| ID NO | Sequence |
| 1 | SYSMN |
| 2 | SISSSSSYIYYADSVKG |
| 3 | RHGYSNSDAFDN |
| 4 | RASQGISHYLV |
| 5 | AASSLQS |
| 6 | LQHNSYPWT |
| 7 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSS |
| 8 | DIQMTQSPSAMSASVGDRVTITCRASQGISHYLVWFQQKPGKVPKRLIYAASSLQSGV |
| | PSRFSGSGSGTEFTLTISSLQPEDFATYYCLQHNSYPWTFGQGTKVEIK |
| 9 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSC |
| 10 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSSASTKGPCVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKTHT |
| | GGGGQGGGGQGGGGQGGGGQGGGGQEVQLLESGGGLVQPGGSLRLSCAASGRYID |
| | ETAVAWFRQAPGKGREFVAGIGGGVDITYYADSVKGRFTISRDNSKNTLYLQMNSLR |
| | PEDTAVYYCGARPGRPLITSKVADLYPYWGQGTLVTVSSPP |
| 11 | DIQMTQSPSAMSASVGDRVTITCRASQGISHYLVWFQQKPGKVPKRLIYAASSLQSGV |
| | PSRFSGSGSGTEFTLTISSLQPEDFATYYCLQHNSYPWTFGQGTKVEIKRTVAAPSVFIF |
| | PPSDEQLKSGTASVVCLLNNFYPREAKVQWKVDNALQSGNSQESVTEQDSKDSTYSL |
| | SSTLTLSKADYEKHKVYACEVTHQGLSSPVTKSFNRGEC |
| 12 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCGGGGQ |
| | GGGGQGGGGQGGGGQGGGGQEVQLLESGGGLVQPGGSLRLSCAASGRYIDETAVA |
| | WFRQAPGKGREFVAGIGGGVDITYYADSVKGRFTISRDNSKNTLYLQMNSLRPEDTA |
| | VYYCGARPGRPLITSKVADLYPYWGQGTLVTVSSPP |
| 13 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSSASTKGPCVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPP |
| | CPAPEAAGGPSVFLFPPKPKDTLMISRTPEVTCVVVDVSQEDPEVQFNWYVDGVEVH |
| | NAKTKPREEQFNSTYRVVSVLTVLHQDWLNGKEYKCKVSNKGLPSSIEKTISKAKGQ |
| | PREPQVSTLPPSQEEMTKNQVSLMCLVYGFYPSDIAVEWESNGQPENNYKTTPPVLD |
| | SDGSFFLYSVLTVDKSRWQEGNVFSCSVMHEALHNHYTQKSLSLSLG |
| 14 | ESKYGPPCPPCPAPEAAGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVSNKGLPSSI |
| | EKTISKAKGQPREPQVYTLPPSQGDMTKNQVQLTCLVKGFYPSDIAVEWESNGQPEN |
| | NYKTTPPVLDSDGSFFLASRLTVDKSRWQEGNVFSCSVMHEALHNHYTQKSLSLSLG |
| 15 | EVQLVESGGGLVKPGGSLRLSCVASGFTFSSYSMNWVRQAPGKGLEWVSSISSSSSYI |
| | YYADSVKGRFTISRDNAKNSLYLQMNSLRAEDTAVYYCARRHGYSNSDAFDNWGQ |
| | GTLVTVSSASTKGPSVFPLAPVSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPP |
| | VPAPEAAGGPSVFLFPPKPKDTLMISRTPEVTCVVVDVSQEDPEVQFNWYVDGVEVH |
| | NAKTKPREEQFNSTYRVVSVLTVLHQDWLNGKEYKCKVSNKGLPSSIEKTISKAKGQ |
| | PREPQVSTLPPSQEEMTKNQVSLMCLVYGFYPSDIAVEWESNGQPENNYKTTPPVLD |
| | SDGSFFLYSVLTVDKSRWQEGNVFSCSVMHEALHNHYTQKSLSLSLG |
| 16 | ESKYGPPCPPVPAPEAAGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVSNKGLPSSI |
| | EKTISKAKGQPREPQVYTLPPSQGDMTKNQVQLTCLVKGFYPSDIAVEWESNGQPEN |
| | NYKTTPPVLDSDGSFFLASRLTVDKSRWQEGNVFSCSVMHEALHNHYTQKSLSLSLG |
| 17 | DIQMTQSPSAMSASVGDRVTITCRASQGISHYLVWFQQKPGKVPKRLIYAASSLQSGV |
| | PSRFSGSGSGTEFTLTISSLQPEDFATYYCLQHNSYPWTFGQGTKVEIKRTVAAPSVFIF |
| | PPSDEQLKSGTASVVCLLNNFYPREAKVQWKVDNALQSGNSQESVTEQDSKDSTYSL |
| | SSTLTLSKADYEKHKVYACEVTHQGLSSPVTKSFNRGEV |
| 18 | ETAVA |
| 19 | GIGGGVDITYYADSVKG |
| 20 | RPGRPLITSKVADLYPY |
| 21 | EVQLLESGGGLVQPGGSLRLSCAASGRYIDETAVAWFRQAPGKGREFVAGIGGGVDI |
| | TYYADSVKGRFTISRDNSKNTLYLQMNSLRPEDTAVYYCGARPGRPLITSKVADLYP |
| | YWGQGTLVTVSSPP |
| 22 | GGGGQGGGGQGGGGQGGGGQ |
| 23 | CUGUACAAGUGCUCAGUUCCA |
| 24 | UGGAACUGAGCACUUGUACAGGA |
| 25 | UUGUACAAGUGCUCAGUUCCA |
| 26 | UGUACAAGUGCUCAGUUCCAA |
| 27 | UUGGAACUGAGCACUUGUACAGG |
| 28 | mC*mU*mGmUmAmCmAmAfGfUfGmCmUmCmAmGmUmUmC*mC*mA* |
| 29 | mU*fG*mGmAfAmCmUfGmAmGmCmAmCfUmUfGmUmAmCmAmG*mG*mA |
| 30 | [NH₂mU]*mU*mGmUmAmCmAmAfGfUfGmCmUmCmAmGmUmUmC*mC*mA |
| 31 | mU*mG*mUmAmCmAmAmGfUfGfCmUmCmAmGmUmUmCmC*mA*mA* |
| 32 | mU*fU*mGmGfAmAmCfUmGmAmGmCmAfCmUfUmGmUmAmCmA*mG*mG |
| 33 | GGCGACGACC AGAAGGGGCC CAAGAGAGGG GGCGAGCGAC CGAGCGCCGC |
| | GACGCGGAAG |
| | TGAGGTGCGT GCGGGCTGCA GCGCAGACCC CGGCCCGGCC CCTCCGAGAG |
| | CGTCCTGGGC |
| | GCTCCCTCAC GCCTTGCCTT CAAGCCTTCT GCCTTTCCAC CCTCGTGAGC |
| | GGAGAACTGG |
| | GAGTGGCCAT TCGACGACAG TGTGGTGTAA AGGAATTCAT TAGCCATGGA |
| | TGTATTCATG |
| | AAAGGACTTT CAAAGGCCAA GGAGGGAGTT GTGGCTGCTG CTGAGAAAAC |
| | CAAACAGGGT |
| | GTGGCAGAAG CAGCAGGAAA GACAAAAGAG GGTGTTCTCT ATGTAGGCTC |
| | CAAAACCAAG |
| | GAGGGAGTGG TGCATGGTGT GGCAACAGTG GCTGAGAAGA CCAAAGAGCA |
| | AGTGACAAAT |
| | GTTGGAGGAG CAGTGGTGAC GGGTGTGACA GCAGTAGCCC AGAAGACAGT |
| | GGAGGGAGCA |
| | GGGAGCATTG CAGCAGCCAC TGGCTTTGTC AAAAAGGACC AGTTGGGCAA |
| | GAATGAAGAA |
| | GGAGCCCCAC AGGAAGGAAT TCTGGAAGAT ATGCCTGTGG ATCCTGACAA |
| | TGAGGCTTAT |
| | GAAATGCCTT CTGAGGAAGG GTATCAAGAC TACGAACCTG AAGCCTAAGA |
| | AATATCTTTG |
| | CTCCCAGTTT CTTGAGATCT GCTGACAGAT GTTCCATCCT GTACAAGTGC |
| | TCAGTTCCAA |
| | TGTGCCCAGT CATGACATTT CTCAAAGTTT TTACAGTGTA TCTCGAAGTC |
| | TTCCATCAGC |
| | AGTGATTGAA GTATCTGTAC CTGCCCCCAC TCAGCATTTC GGTGCTTCCC |
| | TTTCACTGAA |
| | GTGAATACAT GGTAGCAGGG TCTTTGTGTG CTGTGGATTT TGTGGCTTCA |
| | ATCTACGATG |
| | TTAAAACAAA TTAAAAACAC CTAAGTGACT ACCACTTATT TCTAAATCCT |
| | CACTATTTTT |
| | TTGTTGCTGT TGTTCAGAAG TTGTTAGTGA TTTGCTATCA TATATTATAA |
| | GATTTTTAGG |
| | TGTCTTTTAA TGATACTGTC TAAGAATAAT GACGTATTGT GAAATTTGTT |
| | AATATATATA |
| | ATACTTAAAA ATATGTGAGC ATGAAACTAT GCACCTATAA ATACTAAATA |
| | TGAAATTTTA |
| | CCATTTTGCG ATGTGTTTTA TTCACTTGTG TTTGTATATA AATGGTGAGA |
| | ATTAAAATAA |
| | AACGTTATCT CATTGCAAAA ATATTTTATT TTTATCCCAT CTCACTTTAA |
| | TAATAAAAAT |
| | CATGCTTATA AGCAACATGA ATTAAGAACT GACACAAAGG ACAAAAATAT |
| | AAAGTTATTA |
| | ATAGCCATTT GAAGAAGGAG GAATTTTAGA AGAGGTAGAG AAAATGGAAC |
| | ATTAACCCTA |
| | CACTCGGAAT TCCCTGAAGC AACACTGCCA GAAGTGTGTT TTGGTATGCA |
| | CTGGTTCCTT |
| | AAGTGGCTGT GATTAATTAT TGAAAGTGGG GTGTTGAAGA CCCCAACTAC |
| | TATTGTAGAG |
| | TGGTCTATTT CTCCCTTCAA TCCTGTCAAT GTTTGCTTTA CGTATTTTGG |
| | GGAACTGTTG |
| | TTTGATGTGT ATGTGTTTAT AATTGTTATA CATTTTTAAT TGAGCCTTTT |
| | ATTAACATAT |
| | ATTGTTATTT TTGTCTCGAA ATAATTTTTT AGTTAAAATC TATTTTGTCT |
| | GATATTGGTG |
| | TGAATGCTGT ACCTTTCTGA CAATAAATAA TATTCGACCA TGAATAAAAA |
| | AAAAAAAAAA |
| | GTGGGTTCCC GGGAACTAAG CAGTGTAGAA GATGATTTTG ACTACACCCT |
| | CCTTAGAGAG |
| | CCATAAGACA CATTAGCACA TATTAGCACA TTCAAGGCTC TGAGAGAATG |
| | TGGTTAACTT |
| | TGTTTAACTC AGCATTCCTC ACTTTTTTTT TTTAATCATC AGAAATTCTC |
| | TCTCTCTCTC |
| | TCTCTTTTTC TCTCGCTCTC TTTTTTTTTT TTTTTTTACA GGAAATGCCT |
| | TTAAACATCG |
| | TTGGAACTAC CAGAGTCACC TTAAAGGAGA TCAATTCTCT AGACTGATAA |
| | AAATTTCATG |
| | GCCTCCTTTA AATGTTGCCA AATATATGAA TTCTAGGATT TTTCCTTAGG |
| | AAAGGTTTTT |
| | CTCTTTCAGG GAAGATCTAT TAACTCCCCA TGGGTGCTGA AAATAAACTT |
| | GATGGTGAAA |
| | AACTCTGTAT AAATTAATTT AAAAATTATT TGGTTTCTCT TTTTAATTAT |
| | TCTGGGGCAT |
| | AGTCATTTCT AAAAGTCACT AGTAGAAAGT ATAATTTCAA GACAGAATAT |
| | TCTAGACATG |
| | CTAGCAGTTT ATATGTATTC ATGAGTAATG TGATATATAT TGGGCGCTGG |
| | TGAGGAAGGA |
| | AGGAGGAATG AGTGACTATA AGGATGGTTA CCATAGAAAC TTCCTTTTTT |
| | ACCTAATTGA |
| | AGAGAGACTA CTACAGAGTG CTAAGCTGCA TGTGTCATCT TACACTAGAG |
| | AGAAATGGTA |
| | AGTTTCTTGT TTTATTTAAG TTATGTTTAA GCAAGGAAAG GATTTGTTAT |
| | TGAACAGTAT |
| | ATTTCAGGAA GGTTAGAAAG TGGCGGTTAG GATATATTTT AAATCTACCT |
| | AAAGCAGCAT |
| | ATTTTAAAAA TTTAAAAGTA TTGGTATTAA ATTAAGAAAT AGAGGACAGA |
| | ACTAGACTGA |
| | TAGCAGTGAC CTAGAACAAT TTGAGATTAG GAAAGTTGTG ACCATGAATT |
| | TAAGGATTTA |
| | TGTGGATACA AATTCTCCTT TAAAGTGTTT CTTCCCTTAA TATTTATCTG |
| | ACGGTAATTT |
| | TTGAGCAGTG AATTACTTTA TATATCTTAA TAGTTTATTT GGGACCAAAC |
| | ACTTAAACAA |
| | AAAGTTCTTT AAGTCATATA AGCCTTTTCA GGAAGCTTGT CTCATATTCA |
| | CTCCCGAGAC |
| | ATTCACCTGC CAAGTGGCCT GAGGATCAAT CCAGTCCTAG GTTTATTTTG |
| | CAGACTTACA |
| | TTCTCCCAAG TTATTCAGCC TCATATGACT CCACGGTCGG CTTTACCAAA |
| | ACAGTTCAGA |
| | GTGCACTTTG GCACACAATT GGGAACAGAA CAATCTAATG TGTGGTTTGG |
| | TATTCCAAGT |
| | GGGGTCTTTT TCAGAATCTC TGCACTAGTG TGAGATGCAA ACATGTTTCC |
| | TCATCTTTCT |
| | GGCTTATCCA GTATGTAGCT ATTTGTGACA TAATAAATAT ATACATATAT |
| | GAAAATA |
| 34 | MDVFMKGLSK AKEGVVAAAE KTKQGVAEAA GKTKEGVLYV GSKTKEGVVH |
| | GVATVAEKTK |
| | EQVTNVGGAV VTGVTAVAQK TVEGAGSIAA ATGFVKKDQL GKNEEGAPQE |
| | GILEDMPVDP |
| | DNEAYEMPSE EGYQDYEPEA |
| 35 | MMDQARSAFS NLFGGEPLSY TRFSLARQVD GDNSHVEMKL AVDEEENADN |
| | NTKANVTKPK |
| | RCSGSICYGT IAVIVFFLIG FMIGYLGYCK GVEPKTECER LAGTESPVRE |
| | EPGEDFPAAR |
| | RLYWDDLKRK LSEKLDSTDF TGTIKLLNEN SYVPREAGSQ KDENLALYVE |
| | NQFREFKLSK |
| | VWRDQHFVKI QVKDSAQNSV IIVDKNGRLV YLVENPGGYV AYSKAATVTG |
| | KLVHANFGTK |
| | KDFEDLYTPV NGSIVIVRAG KITFAEKVAN AESLNAIGVL IYMDQTKFPI |
| | VNAELSFFGH |
| | AHLGTGDPYT PGFPSFNHTQ FPPSRSSGLP NIPVQTISRA AAEKLFGNME |
| | GDCPSDWKTD |
| | STCRMVTSES KNVKLTVSNV LKEIKILNIF GVIKGFVEPD HYVVVGAQRD |
| | AWGPGAAKSG |
| | VGTALLLKLA QMFSDMVLKD GFQPSRSIIF ASWSAGDFGS VGATEWLEGY |
| | LSSLHLKAFT |
| | YINLDKAVLG TSNFKVSASP LLYTLIEKTM QNVKHPVTGQ FLYQDSNWAS |
| | KVEKLTLDNA |
| | AFPFLAYSGI PAVSFCFCED TDYPYLGTTM DTYKELIERI PELNKVARAA |
| | AEVAGQFVIK |
| | LTHDVELNLD YERYNSQLLS FVRDLNQYRA DIKEMGLSLQ WLYSARGDFF |
| | RATSRLTTDF |
| | GNAEKTDRFV MKKLNDRVMR VEYHFLSPYV SPKESPFRHV FWGSGSHTLP |
| | ALLENLKLRK |
| | QNNGAFNETL FRNQLALATW TIQGAANALS GDVWDIDNEF |
| 36 | MMDQARSAFS NLFGGEPLSY TRFSLARQVD GDNSHVEMKL AADEEENADN |
| | NMKASVRKPK |
| | RFNGRLCFAA IALVIFFLIG FMSGYLGYCK RVEQKEECVK LAETEETDKS |
| | ETMETEDVPT |
| | SSRLYWADLK TLLSEKLNSI EFADTIKQLS QNTYTPREAG SQKDESLAYY |
| | IENQFHEFKF |
| | SKVWRDEHYV KIQVKSSIGQ NMVTIVQSNG NLDPVESPEG YVAFSKPTEV |
| | SGKLVHANFG |
| | TKKDFEELSY SVNGSLVIVR AGEITFAEKV ANAQSFNAIG VLIYMDKNKF |
| | PVVEADLALF |
| | GHAHLGTGDP YTPGFPSFNH TQFPPSQSSG LPNIPVQTIS RAAAEKLFGK |
| | MEGSCPARWN |
| | IDSSCKLELS QNQNVKLIVK NVLKERRILN IFGVIKGYEE PDRYVVVGAQ |
| | RDALGAGVAA |
| | KSSVGTGLLL KLAQVFSDMI SKDGFRPSRS IIFASWTAGD FGAVGATEWL |
| | EGYLSSLHLK |
| | AFTYINLDKV VLGTSNFKVS ASPLLYTLMG KIMQDVKHPV DGKSLYRDSN |
| | WISKVEKLSF |
| | DNAAYPFLAY SGIPAVSFCF CEDADYPYLG TRLDTYEALT QKVPQLNQMV |
| | RTAAEVAGQL |
| | IIKLTHDVEL NLDYEMYNSK LLSFMKDLNQ FKTDIRDMGL SLQWLYSARG |
| | DYFRATSRLT |
| | TDFHNAEKTN RFVMREINDR IMKVEYHFLS PYVSPRESPF RHIFWGSGSH |
| | TLSALVENLK |
| | LRQKNITAFN ETLFRNQLAL ATWTIQGVAN ALSGDIWNID NEF |
| 37 | HHHHHHCKRVEQKEECVKLAETEETDKSETMETEDVPTSSRLYWADLKTLLSEKLN |
| | SIEFADTIKQLSQNTYTPREAGSQKDESLAYYIENQFHEFKFSKVWRDEHYVKIQVKS |
| | SIGQNMVTIVQSNGNLDPVESPEGYVAFSKPTEVSGKLVHANFGTKKDFEELSYSVN |
| | GSLVIVRAGEITFAEKVANAQSFNAIGVLIYMDKNKFPVVEADLALFGHAHLGTGDP |
| | YTPGFPSFNHTQFPPSQSSGLPNIPVQTISRAAAEKLFGKMEGSCPARWNIDSSCKLEL |
| | SQNQNVKLIVKNVLKERRILNIFGVIKGYEEPDRYVVVGAQRDALGAGVAAKSSVGT |
| | GLLLKLAQVFSDMISKDGFRPSRSIIFASWTAGDFGAVGATEWLEGYLSSLHLKAFTY |
| | INLDKVVLGTSNFKVSASPLLYTLMGKIMQDVKHPVDGKSLYRDSNWISKVEKLSFD |
| | NAAYPFLAYSGIPAVSFCFCEDADYPYLGTRLDTYEALTQKVPQLNQMVRTAAEVA |
| | GQLIIKLTHDVELNLDYEMYNSKLLSFMKDLNQFKTDIRDMGLSLQWLYSARGDYFR |
| | ATSRLTTDFHNAEKTNRFVMREINDRIMKVEYHFLSPYVSPRESPFRHIFWGSGSHTLS |
| | ALVENLKLRQKNITAFNETLFRNQLALATWTIQGVANALSGDIWNIDNEF |
| 38 | HHHHHHCKGVEPKTECERLAGTESPVREEPGEDFPAARRLYWDDLKRKLSEKLDST |
| | DFTGTIKLLNENSYVPREAGSQKDENLALYVENQFREFKLSKVWRDQHFVKIQVKDS |
| | AQNSVIIVDKNGRLVYLVENPGGYVAYSKAATVTGKLVHANFGTKKDFEDLYTPVN |
| | GSIVIVRAGKITFAEKVANAESLNAIGVLIYMDQTKFPIVNAELSFFGHAHLGTGDPYT |
| | PGFPSFNHTQFPPSRSSGLPNIPVQTISRAAAEKLFGNMEGDCPSDWKTDSTCRMVTSE |
| | SKNVKLTVSNVLKEIKILNIFGVIKGFVEPDHYVVVGAQRDAWGPGAAKSGVGTALL |
| | LKLAQMFSDMVLKDGFQPSRSIIFASWSAGDFGSVGATEWLEGYLSSLHLKAFTYINL |
| | DKAVLGTSNFKVSASPLLYTLIEKTMQNVKHPVTGQFLYQDSNWASKVEKLTLDNA |
| | AFPFLAYSGIPAVSFCFCEDTDYPYLGTTMDTYKELIERIPELNKVARAAAEVAGQFVI |
| | KLTHDVELNLDYERYNSQLLSFVRDLNQYRADIKEMGLSLOWLYSARGDFFRATSRL |
| | TTDFGNAEKTDRFVMKKLNDRVMRVEYHFLSPYVSPKESPFRHVFWGSGSHTLPALL |
| | ENLKLRKQNNGAFNETLFRNQLALATWTIQGAANALSGDVWDIDNEF |
| 39 | HHHHHHHHGKPIPNPLLGLDSTGGGGSDSAQNSVIIVDKNGRLVYLVENPGGYVAYS |
| | KAATVTGKLVHANFGTKKDFEDLYTPVNGSIVIVRAGKITFAEKVANAESLNAIGVLI |
| | YMDQTKFPIVNAELSFFGHAHLGGGGGGLPNIPVQTISRAAAEKLFGNMEGDCPSDW |
| | KTDSTCRMVTSESKNVKLTVS |

1. A conjugate of Formula (I): A-L-D,
wherein A is an antibody or antibody fragment comprising two cysteine residues,
wherein D is a therapeutic agent,
and wherein L is a linker comprising the following formula:

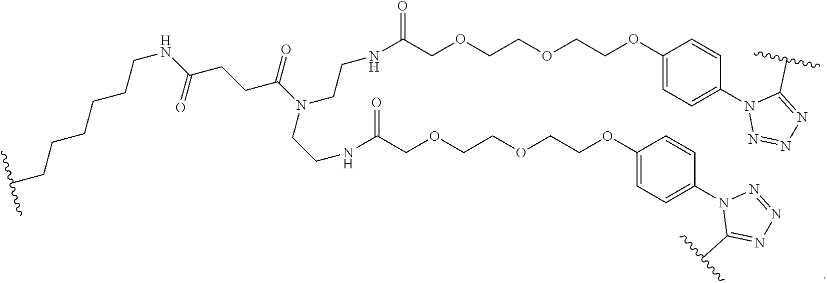 [SMILES: *CCCCCCNC(=O)CCC(=O)N(CCNC(=O)COCCOCCOc1ccc(-n2nnnc2*)cc1)CCNC(=O)COCCOCCOc1ccc(-n2nnnc2*)cc1]

2. The conjugate of claim 1, wherein the conjugate comprises the following formula:

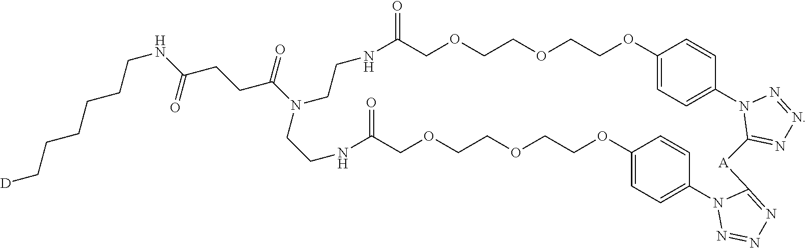

3. The conjugate of claim 1, wherein A is a monoclonal antibody, heterodimeric antibody, one-arm heteromab, Fab, or Fab-VHH.

4. The conjugate of claim 1, wherein A is a human TFR binding antibody or antibody fragment comprising a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6

5. The conjugate of claim 1, wherein A comprises a VH comprising SEQ ID NO: 7 and a VL comprising SEQ ID NO: 8.

6. The conjugate of claim 1, wherein A comprises one heavy chain (HC) and one light chain (LC), wherein HC comprises SEQ ID NO: 9 and LC comprises SEQ ID NO: 11.

7. The conjugate of claim 1, wherein A comprises one heavy chain (HC) and one light chain (LC), and wherein the HC comprises SEQ ID NO: 10 and the LC comprises SEQ ID NO: 11.

8. The conjugate of claim 1, wherein A comprises one heavy chain (HC) and one light chain (LC), and wherein the HC comprises SEQ ID NO: 12 and the LC comprises SEQ ID NO: 11.

9. The conjugate of claim 1, wherein A comprises two heavy chains HC1 and HC2 and one light chain LC1, wherein HC1 comprises SEQ ID NO: 13, LC1 comprises SEQ ID NO: 11, HC2 comprises SEQ ID NO: 14.

10. The conjugate of claim 1, wherein A comprises two heavy chains HC1 and HC2 and one light chain LC1, wherein HC1 comprises SEQ ID NO: 15, LC1 comprises SEQ ID NO: 17, HC2 comprises SEQ ID NO: 16.

11. The conjugate of claim 1, wherein D is an oligonucleotide.

12. The conjugate of claim 1, wherein D is a double stranded RNA (dsRNA) comprising a sense stand and an antisense strand.

13. The conjugate of claim 12, wherein L is connected to the 3′ end of the sense strand.

14. The conjugate of claim 12, wherein L is connected to the 5′ end of the sense strand.

15. The conjugate of claim 12, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A CACNA1B, or PRNP mRNA.

16. The conjugate of claim 15, wherein the antisense strand is complementary to SNCA mRNA.

17. The conjugate of claim 16, wherein the sense strand and the antisense strand comprise a pair of nucleic acid sequences selected from the group consisting of:
(a) the sense strand comprises SEQ ID NO: 23, and the antisense strand comprises SEQ ID NO: 24;
(b) the sense strand comprises SEQ ID NO: 25, and the antisense strand comprises SEQ ID NO: 24; and
(c) the sense strand comprises SEQ ID NO: 26, and the antisense strand comprises SEQ ID NO: 27,
wherein optionally one or more nucleotides of the sense strand and the antisense strand are independently modified nucleotides, and wherein optionally one or more internucleotide linkages of the sense strand and the antisense strand are modified internucleotide linkages.

18. The conjugate of claim 17, wherein one or more nucleotides of the sense strand are modified nucleotides.

19. The conjugate of claim 18, wherein each nucleotide of the sense strand is a modified nucleotide.

20. The conjugate of claim 17, wherein one or more nucleotides of the antisense strand are modified nucleotides.

21. The conjugate of claim 20, wherein each nucleotide of the antisense strand is a modified nucleotide.

22. The conjugate of claim 17, wherein the modified nucleotide is a 2′-fluoro modified nucleotide, 2′-O-methyl modified nucleotide or 2′-O—C₁₆ alkyl modified nucleotide.

23. The conjugate of claim 17, wherein the sense strand and the antisense strand have one or more modified internucleotide linkages.

24. The conjugate of claim 23, wherein the modified internucleotide linkage is phosphorothioate linkage.

25. The conjugate of claim 24, wherein the sense strand has four or five phosphorothioate linkages.

26. The conjugate of claim 24, wherein the antisense strand has four or five phosphorothioate linkages.

27. The conjugate of claim 17, wherein the antisense strand has a phosphate analog at 5′ end.

28. The conjugate of claim 27, wherein the phosphate analog is 5′-vinylphosphonate.

29. The conjugate of claim 17, wherein the sense strand or the antisense strand comprises an abasic moiety or inverted abasic moiety.

30. The conjugate of claim 17, wherein the sense strand and the antisense strand comprise a pair of nucleic acid sequences selected from the group consisting of:
(a) the sense strand comprises SEQ ID NO: 28, and the antisense strand comprises SEQ ID NO: 29;
(b) the sense strand comprises SEQ ID NO: 30, and the antisense strand comprises SEQ ID NO: 29; and
(c) the sense strand comprises SEQ ID NO: 31, and the antisense strand comprises SEQ ID NO: 32.

31. The conjugate of claim 17, wherein the sense strand and the antisense strand have a pair of nucleic acid sequences selected from the group consisting of:
(a) the sense strand consists of SEQ ID NO: 28, and the antisense strand consists of SEQ ID NO: 29;
(b) the sense strand consists of SEQ ID NO: 30, and the antisense strand consists of SEQ ID NO: 29; and
(c) the sense strand consists of SEQ ID NO: 31, and the antisense strand consists of SEQ ID NO: 32.

32. A pharmaceutical composition comprising the conjugate of claim 1 and a pharmaceutically acceptable carrier.

33. A pharmaceutical composition comprising the conjugate of claim 17 and a pharmaceutically acceptable carrier.

34. A method of treating a neurodegenerative disease in a patient in need thereof, the method comprising administering to the patient an effective amount of the conjugate of claim 1.

35. A method of treating a neurodegenerative disease in a patient in need thereof, the method comprising administering to the patient an effective amount of the conjugate of claim 17.

36. The method of claim 35, wherein the neurodegenerative disease is Parkinson's disease, Alzheimer's disease, multiple system atrophy, or Lewy body dementia.

37. The method of claim 35, wherein the conjugate is administered to the patient intravenously or subcutaneously.

38. A compound comprising the following formula: 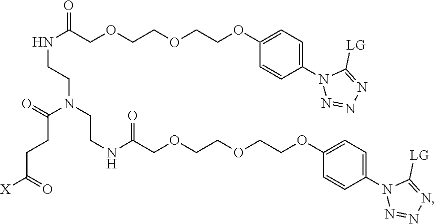
wherein LG is a leaving group and X is an amide coupling partner.

39. The compound of claim 38, wherein the compound comprises the following formula:

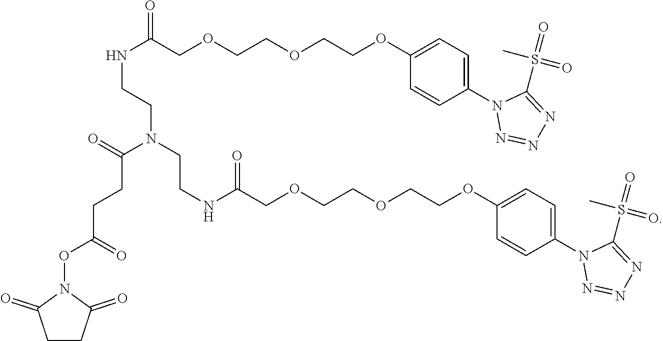 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]

40. A method of generating a conjugate, the method comprising reacting an antibody or antibody fragment comprising two cysteine residues with a compound comprising any one of the following formulae: 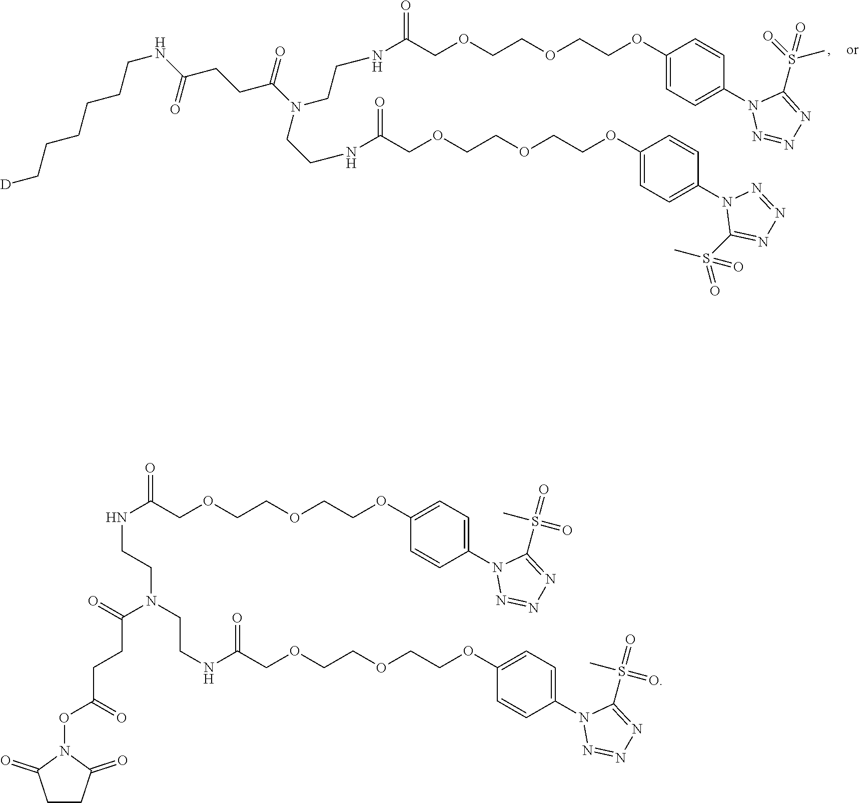 [SMILES: [2H]CCCCCCNC(=O)CCC(=O)N(CCNC(=O)COCCOCCOc1ccc(-n2nnnc2S(C)(=O)=O)cc1)CCNC(=O)COCCOCCOc1ccc(-n2nnnc2S(C)(=O)=O)cc1 ; CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)ON2C(=O)CCC2=O)cc1]


SEQUENCE LISTING (39 sequences)

SEQ ID NO: 1 - protein, 5 aa, synthetic construct
SYSMN

SEQ ID NO: 2 - protein, 17 aa, synthetic construct
SISSSSSYIYYADSVKG

SEQ ID NO: 3 - protein, 12 aa, synthetic construct
RHGYSNSDAFDN

SEQ ID NO: 4 - protein, 11 aa, synthetic construct
RASQGISHYLV

SEQ ID NO: 5 - protein, 7 aa, synthetic construct
AASSLQS

SEQ ID NO: 6 - protein, 9 aa, synthetic construct
LQHNSYPWT

SEQ ID NO: 7 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 8 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 9 - protein, 224 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - protein, 382 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 11 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 377 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 13 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 14 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 15 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPVSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPVPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 16 - protein, 228 aa, synthetic construct
ESKYGPPCPPVPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 17 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 18 - protein, 5 aa, synthetic construct
ETAVA

SEQ ID NO: 19 - protein, 17 aa, synthetic construct
GIGGGVDITYYADSVKG

SEQ ID NO: 20 - protein, 17 aa, synthetic construct
RPGRPLITSKVADLYPY

SEQ ID NO: 21 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 22 - protein, 20 aa, synthetic construct
GGGGQGGGGQGGGGQGGGGQ

SEQ ID NO: 23 - RNA, 21 nt, synthetic construct
  source 1..21: mol_type = other RNA
ctgtacaagtgctcagttcca

SEQ ID NO: 24 - RNA, 23 nt, synthetic construct
  source 1..23: mol_type = other RNA
tggaactgagcacttgtacagga

SEQ ID NO: 25 - RNA, 21 nt, synthetic construct
  source 1..21: mol_type = other RNA
ttgtacaagtgctcagttcca

SEQ ID NO: 26 - RNA, 21 nt, synthetic construct
  source 1..21: mol_type = other RNA
tgtacaagtgctcagttccaa

SEQ ID NO: 27 - RNA, 23 nt, synthetic construct
  source 1..23: mol_type = other RNA
ttggaactgagcacttgtacagg

SEQ ID NO: 28 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate
  source 1..21: mol_type = other RNA
ctgtacaagtgctcagttcca

SEQ ID NO: 29 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = um
  modified_base 8: mod_base = OTHER; note = 2'-fluoroguanosine
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 30 - RNA, 21 nt, synthetic construct
  misc_feature 1..21: note = /note="Description of Artificial Sequence: Synthetic oligonucleotide"
  modified_base 1: mod_base = OTHER; note = 2'-O-methyluridine with a 3'-phosphorothioate linkage and a 5'-amino group
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ttgtacaagtgctcagttcca

SEQ ID NO: 31 - RNA, 21 nt, synthetic construct
  misc_feature 1..21: note = /note="Description of Artificial Sequence: Synthetic oligonucleotide"
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate
  source 1..21: mol_type = other RNA
tgtacaagtgctcagttccaa

SEQ ID NO: 32 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = cm
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
  source 1..23: mol_type = other RNA
ttggaactgagcacttgtacagg

SEQ ID NO: 33 - RNA, 3177 nt, Homo sapiens
  source 1..3177: mol_type = mRNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 34 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 37 - protein, 681 aa, synthetic construct
HHHHHHCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 38 - protein, 678 aa, synthetic construct
HHHHHHCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 39 - protein, 193 aa, synthetic construct
HHHHHHHHGKPIPNPLLGLDSTGGGGSDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGGGGGGLPNIPVQTISRAAAEKLFGNMEGDCPSDWKTDSTCRMVTSESKNVKLTVS